SEQUENCE LISTING (15 sequences)

SEQ ID NO: 1 - DNA, 336 bp, Candida Albicans
atgaaattctccaccactttattagctttaaccgcctcaattgctgctgttatgtctgctgattcatcagctgctgcctctggtgctgcctcggctgcttctggtgccaaatctggtgctacctcagctgcttctggtgccaaatccggtgcttcttcagttgcttctgccgctaaatctggtgtttcttcagctgcctcagctgctaaatctggtgcttcatctgctaccggtggttcatctgctgccaaatctggctcatcaagtggtgccggttttgctcctgtcgctggtgctggtagcttggcagccattgctggtcttttgttgttgtaa

SEQ ID NO: 2 - protein, 111 aa, Candida Albicans
MKFSTTLLALTASIAAVMSADSSAAASGAASAASGAKSGATSAASGAKSGASSVASAAKSGVSSAASAAKSGASSATGGSSAAKSGSSSGAGFAPVAGAGSLAAIAGLLLL

SEQ ID NO: 3 - DNA, 507 bp, Candida Albicans
atgagattcgctttcacaactgtatcattatcccttttattgtcttctttagttgcttcagaagctgcatcatccgatgttcaattcttgactgctttggtaggtgattatcaagatcataagaccgattatattaaattttttgccaccgcaaaagatgttccaggtgatttatctacgttggctaccaaagtgttgacttatactgatgattcatacacaactttgttgaatgatgattctttgaatgtttccaacttagaagcatatgctactagtttgccatggtattccagaattcaagctgatgctggtggcaaaggttctgcctccggttctgcctctggctctggttctgccaaatcaactgcaagtgctgaaaaatctagtggctcaagtgcttctgcttcaagcactgcaggtggttcctcttctaaaggtggtgtaagtgaacttgttgcccctgttggtgctgttgttggtgctttggcagttgctttaatgtaa

SEQ ID NO: 4 - protein, 168 aa, Candida Albicans
MRFAFTTVSLSLLLSSLVASEAASSDVQFLTALVGDYQDHKTDYIKFFATAKDVPGDLSTLATKVLTYTDDSYTTLLNDDSLNVSNLEAYATSLPWYSRIQADAGGKGSASGSASGSGSAKSTASAEKSSGSSASASSTAGGSSSKGGVSELVAPVGAVVGALAVALM

SEQ ID NO: 5 - DNA, 1682 bp, Candida Albicans
atgataatctttcggaaatcttttttcactttttggcttttgcttaattctgtcttagctcttgttatcactcaaaacagagtcgatcgtggtgttcttgacgttagtgttggaaatatcaccatcaattctggagcttcttggtcaattatcaacaatgctatatcaacccttgttggaagtttaactgttcagcccaatgctggtctttacattaccctgacttcaccccttttgtcacttcaagtcacattaacttctttgcttagcacaattcaaaacaatggtattattgcgttcaattccctgccttccttaacatcgtccacatataatttagttggtttatcccttgtcaacactggagaaatgtatttttctgcttctggtgttttacctagtgttatggctcttactgctgcatcttggtcaaacagtggattgatggcattttatcaaaatcaaagaagttctggtattgttagtcttgggacaccatcaggttcaataaccaataatggtcaaatctgtttgattaacgaagtctacaaacagaccacaagcatcaacggttctggttgtttcactgccaatcgtaactcgacaatatatattgccaatgtattgttaccagtttccacatcgcaaaatttttatttggcagacagccaatcttccataattgttcaagctatttcaacccctcaagtgtttaatgtctatgggtttggtaacggtaatatggtcggggttactcttccattgatcggtaatatatggaatccagcatatagttataatccatccacaggtattttaagattgagaaatttttttgtgtatcaagattttaatattggtcctggttataatcctagtttatttctgatcgttactgacaatggtgctggtcttccctcaacaatactcggttcggtttcttatagtggtcctgttccaccaagagctttacccgcatcttgtaagattgcatgtaaacccgtgcctaccgcgccaggaactaatccaaccgagtacacgaccacaataacaacaacaaattctgctggtaagccattgacagaaactggtgtggttgatattctgactgatagtaacggatcatggttctcaagtactacaatctttccaacttcgtcgtcaagtagtagcagtagcagcactgtttcttcaactgctccgtcatcctcaagcaccaaaccttcatccagtagccaaccatcttctactccaccaccatcttcaagtagtaaagcatcatcaactactccaagctctagtagtcaatcgtcttcaactactccaagctcaagcagtaagccttcctcaactgtaccaccaactggcagcagtcagtcatcttcaactatcccaagttccagtactcaaccttcttctactgctccatcatctttaagttctccatcttcttctactactccaagctccagcagtcaatcttcattttctgctcaaagctctattggccagacatcgtcttctactgtatcttcgtcgagtagtcaaccatcgtgctgggagtcatcaagcagtcagtcgtcatccggtacgacaagttccagtagtcagttttcttcaagtgccccaccgtcaagtacacaatctctgtttactgctgaaagctccaatagtcaattatc

SEQ ID NO: 6 - protein, 560 aa, Candida Albicans
MIIFRKSFFTFWLLLNSVLALVITQNRVDRGVLDVSVGNITINSGASWSIINNAISTLVGSLTVQPNAGLYITSTSPLLSLQVTLTSLLSTIQNNGIIAFNSSPSLTSSTYNLVGLSLVNTGEMYFSASGVLPSVMALTAASWSNSGLMAFYQNQRSSGIVSLGTPSGSITNNGQICLINEVYKQTTSINGSGCFTANRNSTIYIANVLLPVSTSQNFYLADSQSSIIVQAISTPQVFNVYGFGNGNMVGVTLPLIGNIWNPAYSYNPSTGILRLRNFFVYQDFNIGPGYNPSLFSIVTDNGAGLPSTILGSVSYSGPVPPRALPASCKIACKPVPTAPGTNPTEYTTTITTTNSAGKPLTETGVVDISTDSNGSWFSSTTIFPTSSSSSSSSSTVSSTAPSSSSTKPSSSSQPSSTPPPSSSSKASSTTPSSSSQSSSTTPSSSSKPSSTVPPTGSSQSSSTIPSSSTQPSSTAPSSLSSPSSSTTPSSSSQSSFSAQSSIGQTSSSTVSSSSSQPSCWESSSSQSSSGTTSSSSQFSSSAPPSSTQSSFTAESSNSQL

SEQ ID NO: 7 - DNA, 30 bp, Candida Albicans
aagggccccacaaaataaaagcagcaggaa

SEQ ID NO: 8 - DNA, 29 bp, Candida Albicans
ccgctcgagttccaactttaatcccgcac

SEQ ID NO: 9 - DNA, 36 bp, Candida Albicans
ataagaatgcggccgcgtgccaccagtcaaattcaa

SEQ ID NO: 10 - DNA, 27 bp, Candida Albicans
cgagctcccgaaatgccaccatagttt

SEQ ID NO: 11 - DNA, 29 bp, Candida Albicans
aagggcccgtgcggggattaaagttggaa

SEQ ID NO: 12 - DNA, 29 bp, Candida Albicans
ccgctcgagttgttgttgtaagcgaagcc

SEQ ID NO: 13 - DNA, 36 bp, Candida Albicans
ataagaatgcggccgctgaatgagaatgagggggac

SEQ ID NO: 14 - DNA, 27 bp, Candida Albicans
cgagctcttgaatttgacgggtggcaa

SEQ ID NO: 15 - DNA, 29 bp, Candida Albicans
ccgctcgagccgaaatgccaccatagttt

(19) United States
(12) Patent Application Publication
(10) Pub. No.: US 2007/0196378 A1
(43) Pub. Date: Aug. 23, 2007

(54) Hyphae-Specific Cell Wall Proteins Of Candida

(75) Inventors: Henrike Lotz, Stuttgart (DE); Herwig Brunner, Stuttgart (DE); Steffen Rupp, Stuttgart (DE)

Correspondence Address: OSTROLENK FABER GERB & SOFFEN, 1180 AVENUE OF THE AMERICAS, NEW YORK, NY, 100368403, US

(73) Assignee: FRAUNHOFER-GESELLSCHAFT ZUR FORDERUNG DER ANGEWANDTEN FORSCHUNG E.V., München (DE)

(21) Appl. No.: 10/593,213

(22) PCT Filed: Mar. 15, 2005
(86) PCT No.: PCT/EP05/02748

§ 371 Date: Jan. 12, 2007

(30) Foreign Application Priority Data
Mar. 16, 2004 (DE) 10 2004 013 826.5

(51) Int. Cl. A61K 48/00; C12Q 1/68; G01N 33/569; C07H 21/04; A61K 39/395; C12N 9/48; C12N 1/18; C12N 1/16; C12N 15/74

(52) U.S. Cl. 424/151.1; 435/7.31; 435/69.3; 435/212; 435/254.23; 435/254.3; 435/483; 536/23.2; 514/44; 435/6; 530/388.5

(57) ABSTRACT

Nucleic acids that code a cell wall protein required for the hyphal development of Candida; fragments of the nucleic acids; vectors containing the nucleic acids or nucleic acid fragments; host cells containing the vectors; cell wall proteins coded by the nucleic acids; antibodies directed against the proteins; methods for producing the cell wall proteins; methods for characterizing and/or for detecting the hyphal stage of Candida; methods for detecting a Candida infection; methods for identifying substances having a therapeutic effect against diseases caused by Candida; diagnostic and pharmaceutical compositions containing the nucleic acid, nucleic acids fragments, vectors, host cells, proteins and/or antibodies directed against the cell wall protein; kits for conducting in-vitro indentation of the cell wall proteins, and; to the use of the nucleic acids, vectors, proteins or antibodies for diagnosing and treating Candida-caused diseases, i.e., producing diagnostic or pharmaceutical compositions.

The present invention relates to nucleic acids which encode a cell wall protein necessary for the hyphae development of Candida, fragments of the nucleic acids, vectors containing the nucleic acids or nucleic acid fragments, host cells containing the vectors, the cell wall proteins encoded by the nucleic acids, antibodies directed against the proteins, methods for the production of the cell wall proteins, methods for the characterization and/or for the detection of the hyphae stage of Candida, methods for the detection of a Candida infection, methods for the identification of substances having therapeutic action against diseases caused by Candida, diagnostic and pharmaceutical compositions which contain the nucleic acids, nucleic acid fragments, vectors, host cells, proteins and/or antibodies against the cell wall protein, kits for the in vitro identification of the cell wall proteins, and the use of the nucleic acids, vectors, proteins or antibodies for the diagnosis and treatment of diseases caused by Candida or for the production of diagnostic or pharmaceutical compositions.

Candida albicans is the most frequent causative organism of systemic mycoses in humans. Normally, C. albicans, as a commensal organism, colonizes the surfaces of the gastrointestinal tract and the mucous membranes of humans and animals. During a systemic infection, C. albicans, however, can attack a multiplicity of important organs of an organism. In immunosuppressed patients, C. albicans can frequently cause opportunistic infections. In particular in immunosuppressed patients, Candida infections can take an extremely serious course and lead to life-threatening conditions. The incidence of invasive and difficult to treat Candida mycoses is markedly increasing worldwide. The most important risk factors for the development of fungal infections such as Candida mycoses include neutropenia, aggressive tumor therapy, immunosuppression, AIDS, antibiotic therapies, diabetes mellitus, a very long residence time of intravenous catheters, graft-versus-host disease and surgical interventions.

The exact diagnosis of fungal infections, in particular of Candida infections, is extremely difficult, since the clinical findings in the case of fungal infections are predominantly uncharacteristic. For the diagnosis of invasive fungal infections, hitherto mainly cultures have been prepared for the exact species determination, diagnostic tests based on cultures, however, in general not being sufficiently accurate and sensitive. Moreover, it is also very difficult to perform a differentiation between a colonization and an invasion of the fungal pathogen. For the diagnosis of fungal infections, samples from the patient are also examined microscopically. Especially in the presence of only a few hyphae fragments, however, a species differentiation and therefore also an accurate species determination is not always possible. For the detection of invasive fungal infections, in particular Candida infections, serological methods are also employed, it having emerged here, however, that the antibody tests available at present in some cases only react in a reduced manner in immunosuppressed patients, which makes an interpretation of the titer kinetics difficult. In recent years, methods for the detection of fungus-specific DNA with the aid of hybridization methods and the polymerase chain reaction (PCR) were moreover developed which, however, exhibit too low a sensitivity and specificity in clinical use. In particular, it is problematical that the DNA extraction and amplification methods employed at present do not allow routine screening of patients. Overall, it is seen that at present a reliable diagnosis of Candida infections is very difficult, since the diagnosis methods are not sufficiently sensitive or specific, it usually only being possible to detect invasive fungal infections relatively late.

The clinical treatment of C. albicans infections is at present mainly carried out using polyene antibiotics, azole derivatives, alylamines/thiocarbamates, fluoropyrimidines and equinocandines. The antimycotics used at present, however, only partly take into account the medicinal requirements. Azoles, for example ketoconazole, are highly toxic and have a number of undesirable side effects. It has also been shown that resistance develops to an increasing extent against the azoles and equinocandines employed (Schuetzer-Muehlbauer et al., Mol. Microbiol., 48 (2003), 223-235). On account of their high toxicity, polyene macrolides such as amphotericin B and nystatin likewise lead to severe side effects.

The development of further improved diagnostics which allow a reliable assignment of an illness to the infections caused by the genus Candida, and further improved antimycotics for the treatment of infections or diseases which are caused by Candida, is therefore urgently necessary.

The technical problem underlying the present invention therefore consists in the provision of agents and methods which make possible an exact and rapid diagnosis of the diseases and infections caused by Candida, in particular C. albicans, and in the provision of agents and methods which can be employed for the development of novel highly active therapeutics which, in particular, make possible a targeted therapy of infections caused by Candida and which do not have the disadvantages known in the prior art, for example cause no side effects in the organism treated.

The invention solves the technical problem underlying it by the provision of a nucleic acid molecule which encodes a cell wall protein necessary for the hyphae development of a pathogenic fungal organism, selected from the group consisting of:
a) a nucleic acid molecule having one of the nucleodide sequences shown in SEQ ID No. 1, SEQ ID No. 3 or SEQ ID No. 5,
b) a nucleic acid molecule having a nucleotide sequence which encodes a protein having one of the amino acid sequences shown in SEQ ID No. 2, SEQ ID No. 4 or SEQ ID No. 6,
c) a nucleic acid molecule having a nucleotide sequence which over its entire length shows a homology of at least 80% to a nucleotide sequence of one of the nucleic acid molecules of a) or b), and
d) a nucleic acid molecule having a nucleotide sequence which is complementary to a nucleotide sequence of one of the nucleic acid molecules of a) to c).

The present invention thus solves the technical problem underlying it by the provision of nucleic acids which encodes the hyphae-specific cell wall proteins of Candida. The hyphae-specific cell wall proteins Rbr1p, Rbr2p and Rbr3p provided for the first time according to the invention are pH− and/or temperature-regulated virulence factors of C. albicans. Rbr1p, Rbr2p and Rbr3p are necessary for a successful infection of the host by Candida. This was demonstrated by the inventors of the present invention, in particular as exemplified by Rbr1p in a mouse model of systemic candidosis. Investigations of the inventors of the invention show that Rbr1p, Rbr2p and Rbr3p are expressed as a function of the pH in the cell. In particular, it was shown for Rbr1p that it is a GPI-anchored cell wall protein. The hyphae-specific cell wall proteins Rbr1p, Rbr2p and Rbr3p are, as constituents of the cell wall, particularly highly suitable for the development of antimycotics, since cell wall proteins are freely accessible, in particular to small active compound molecules, these not having to get over any cell membrane. By the identification of Rbr1p, Rbr2p and Rbr3p as essential factors for a systemic Candida infection, novel targets still not described up to now are provided, which are particularly suitable for antimycotic development.

Since the cell wall of Candida is responsible for the adhesion of Candida cells to cells of the host organism, the constituents of the cell wall hyphal cells, that is also Rbr1p, Rbr2p and Rbr3p, are of particular importance for the development of the pathogenicity of C. albicans. Since the proteins Rbr1p, Rbr2p and Rbr3p according to the invention only occur in the cell wall of the virulent hyphae form, these cell wall proteins can also be employed according to the invention as serodiagnostic markers, in order, for example, to differentiate an invasive Candida mycosis from a simple Candida colonization, the presence of a Candida infection being indicated by the detection of the inventive Rbr1p, Rbr2p and/or Rbr3p protein in a sample to be investigated.

According to the invention, the Rbr1p, Rbr2p and Rbr3p proteins can advantageously also be employed for the development of diagnostic and/or therapeutic antibodies, with the aid of which a species-specific diagnosis or therapy is possible. All constituents of the cell wall of Candida cells play an essential role in the triggering and modulation of the immune reactions of the host organism directed against Candida. In general, the Candida cell wall consists of complex glucose polymers, for example β-1,3-glucans and β-1,6-glucans, chitin and mannoproteins. Antibodies directed against these antigens therefore always also detect these carbohydrate radicals. Since the mannans of the individual Candida species, however, have high similarity, cross-reactivity exists between the Candida species, such that a differentiation of individual Candida species using antibodies which are directed against glucose polymers is frequently not possible. Antibodies directed against the cell wall proteins Rbr1p, Rbr2p and Rbr3p according to the invention, however, only detect protein-specific epitopes, but not nonspecific mannan constituents.

In connection with the present invention, the term “Candida” includes blastomycetes of the Fungi imperfecti. Blastomycetes of Candida are pleomorphic organisms whose life cycle are characterized by reversible morphogenetic transitions between budding and pseudohyphal or hyphal growth forms. The transition of separate yeast cells (blastospores) to filamentous forms, that is to say hyphae and pseudohyphae, is designated as dimorphism. Genes or the proteins encoded by these genes, which are expressed during hyphal growth, are of enormous importance for the development of the virulence properties of Candida. The transition between filamentous growth and virulence properties is expressed, for example, in that forms of C. albicans which form no hyphae are avirulent in an animal model system, for example Mus musculus (Lo et al., Cell, 90 (1997), 939-949). In addition to C. albicans, these optionally pathogenic fungi also include C. tropicalis, C. krusei, C. parapsilosis, C. guilliermondii, C. glabrata, C. dubliniensis, C. lusitaniae, Trichosporon species or Blastoschizomyces species.

“Differentiation stages” or “growth stages” of Candida cells are understood according to the invention as meaning the morphogenetic growth forms occurring in the Candida life cycle, a differentiation in particular being made between the yeast growth stage and the hyphae and pseudohyphae growth stage. The transition from yeast growth to filamentous growth, that is to say formation of hyphae or pseudohyphae, is designated as dimorphism. The nonvirulent yeast stage is characterized by the occurrence of “blastospores”, which are gram-positive acapsular budding cells of oval to roundish shape, and proliferation by means of budding. The virulent hyphae and pseudohyphae growth stage is characterized by the formation of true mycelium or pseudomycelium. Using the nucleic acid molecules according to the invention, which encode a cell wall protein expressed during hyphae growth, it is thus possible to differentiate the hyphae stage of Candida cells from the yeast stage and therefore to characterize and/or to detect the hyphae stage, the hyphae stage being characterized in that the cell wall protein Rbr1p according to the invention and/or the cell wall protein Rbr2p according to the invention and/or the cell wall protein Rbr3p according to the invention is/are present.

The nucleic acid molecules according to the invention are thus protein-encoding nucleic acids which encode a cell wall protein necessary for the hyphae development of a pathogenic fungal organism, in particular the cell wall protein Rbr1p, the cell wall protein Rbr2p and/or the cell wall protein Rbr3p. In particular, they are nucleic acid molecules which have the sequence of the RBR1 gene, of the RBR2 gene and/or of the RBR3 gene of C. albicans. The RBR1, RBR2 and RBR3 genes are pH− and/or temperature-regulated genes, whose expression is activated by the repressor Nrg1p and repressed by the transcription factor Rim101p.

A further embodiment of the present invention relates to nucleic acid molecules which encode a hyphae-specific cell wall protein of a Candida species, the nucleic acid molecules having a very great homology to the nucleic acids which encode the Rbr1p, Rbr2p or Rbr3p protein of C. albicans over their entire length, the homology being more than 75%, in particular more than 80%, preferably more than 85%, particularly preferably more than 90% and most preferably more than 95% or more than 98%. In connection with the present invention, “homology” is understood as meaning the degree of identity which exists between the nucleotide sequences of two nucleic acid molecules or between the amino acid sequences of two protein molecules. Homologous nucleic acids can be hybridized with the nucleic acid sequence shown in SEQ ID No. 1, SEQ ID No. 3 or SEQ ID No. 5. Hybridization in the context of the invention means hybridization under preferably stringent hybridization conditions, such as are described in Sambrook et al., “Molecular cloning: A laboratory manual”, Cold Spring Harbor Laboratory Press, 2nd edition (1989).

The nucleic acid molecule according to the invention can encode both a wild-type cell wall protein, for example Rbr1p from C. albicans having the amino acid sequence shown in SEQ ID No. 2, Rbr2p form C. albicans having the amino acid sequence shown in SEQ ID No. 4 or Rbr3p from C. albicans having the amino acid sequence shown in SEQ ID No. 6, and a mutated cell wall protein or a derivative thereof. Nucleic acids which encode a mutated cell wall protein compared with Rbr1p, Rbr2p or Rbr3p of C. albicans or a derivative thereof have a nucleic acid sequence which compared with the nucleic acid sequence shown in SEQ ID No. 1, SEQ ID No. 3 or SEQ ID No. 5 is modified, that is to say mutated, in at least one, preferably two or more positions. “Two or more positions” according to the invention means that 2 to at most 150 positions, in particular 2 to at most 120 positions, preferably 2 to at most 90 positions, particularly preferably 2 to at most 60 positions, and most preferably 2 to at most 30 positions, 2 to at most 24 positions, 2 to at most 18 positions, 2 to at most 12 positions or 2 to at most 0.6 positions in the nucleic acid sequence are mutated, the homology of the nucleic acids containing modified positions over their entire length to the nucleic acid sequence shown in SEQ ID No. 1, SEQ ID No. 3 or SEQ ID No. 5 being more than 75%, in particular more than 80%, preferably more than 85%, particularly preferably more than 90% and most preferably more than 95%, 96%, 97%, 98% or 99%.

The differences compared with the nucleic acid sequence shown in SEQ ID No. 1, SEQ ID No. 3 or SEQ ID No. 5, that is to say the mutations, can be caused, for example, by deletions, substitutions, insertions, additions, nucleotide replacements and/or recombination. The mutations can be naturally occurring variations, for example sequences from other, closely related organisms or other clinical isolates of the same Candida species or mutations originating naturally. They can, however, also be mutations which have been artificially produced by technical means, for example by UV rays, X-rays or chemical agents. They can, however, also be sequence modifications which have been produced by means of genetic engineering methods. According to the invention, the nucleic acid molecules can also contain those mutations or modifications of the nucleic acid according to the invention which have been produced by fusion with another gene or areas of such a gene from the same Candida species to another Candida species or another organism. For example, they can be nucleic acid molecules which contain areas of the RBR1 gene according to the invention in combination with areas of the RBR2 gene according to the invention and/or areas of the RBR3 gene according to the invention.

The nucleic acid according to the invention or the nucleic acid molecule according to the invention can, however, also contain a nucleotide sequence which is truncated compared with the nucleotide sequence shown in SEQ ID No. 1, SEQ ID No. 3 or SEQ ID No. 5. Such a truncated nucleic acid molecule can likewise encode a complete cell wall protein Rbr1p, Rbr2p or Rbr3p or else only a fragment thereof, for example a protein domain or an epitope thereof. Independently of whether the truncated nucleic acid molecule used according to the invention encodes a complete cell wall protein Rbr1p, Rbr2p or Rbr3p or only a fragment thereof or not, it must in each case be able to inhibit, in antisense orientation, the protein expression of the nucleic acid encoding the cell wall protein Rbr1p, Rbr2p or Rbr3p, for example a nucleic acid encoding the wild-type protein or a mutated protein, in its natural cellular environment or in a suitable host system. “Inhibition” in the context of the present invention means either a complete blocking or at least a partial reduction of the transcription and/or translation of the nucleic acid to be inhibited.

In a further embodiment, the nucleic acid molecule according to the invention is a molecule having a nucleotide sequence which is complementary to a nucleotide sequence of one of the aforementioned nucleic acid molecules. A nucleic acid or a nucleotides sequence is “complementary” to another nucleic acid or nucleotide sequence if it can form a double strand with this over its entire length in which all nucleotides are present paired by hydrogen bonding according to the rules of Watson and Crick. For example an mRNA molecule is complementary to one of the DNA strands of a gene by which it is encoded.

According to the invention, it is in particular provided that the nucleic acid molecules according to the invention are present in isolated and purified form. According to the invention, it is further provided that the nucleic acid molecule is present as a DNA, RNA, PNA or LNA molecule or as a mixed form thereof. “PNA” (“Peptide Nucleic Acid” or “Polyamide Nucleic Acid”) sequences are molecules which are not negatively charged and act in an identical manner to DNA (Nielsen et al., Science, 254 (1991), 1497-1500; Nielsen et al., Biochemistry, 26 (1997), 5072-5077; Weiler et al., Nuc. Acids Res., 25 (1997), 2792-2799). PNA sequences comprise a polyamide parent structure of N-(2-aminoethyl)glycine units and have no glucose units and no phosphate groups. The different bases are bonded to the parent structure via methylene-carbonyl bonds. “LNA” (Locked Nucleic Acid) molecules comprise the furanose ring conformation restricted by a methylene linker which connects the 2′-O-position to the 4′-C-position. LNAs are incorporated into nucleic acids, for example DNA or RNA, as individual nucleotides. LNA molecules are subject, also like PNA molecules, to the Watson-Crick base pairing rules and hybridize with complementary DNA and/or RNA molecules. LNA/DNA or LNA/RNA duplex molecules show increased thermal stability compared with similar duplex molecules which are exclusively formed from DNA or RNA.

The nucleic acid molecules employed according to the invention can have been isolated from a natural source, preferably from cells of C. albicans or another Candida species. The nucleic acid molecules according to the invention can, however, also have been chemically synthesized by means of a known method.

A further embodiment of the present invention relates to a fragment of a nucleic acid molecule that encodes a cell wall protein necessary for the hyphae development of a pathogenic fungal organism, in particular the cell wall protein Rbr1p, Rbr2p or Rbr3p of C. albicans. A “fragment” of a nucleic acid molecule is understood as meaning a subarea of a nucleic acid molecule according to the invention which in the natural cellular environment of the nucleic acid molecule according to the invention or in a suitable host system can inhibit the protein expression of the nucleic acid according to the invention which encodes a cell wall protein. “Inhibition” in the context of the present invention means either a complete blocking or at least a partial reduction of the transcription and/or translation of the nucleic acid to be inhibited. A nucleic acid or its mRNA present in antisense orientation is complementary to the mRNA of a nucleic acid to be inhibited and bonds with this to form a double-stranded nucleic acid molecule which is enzymatically degraded. In a particularly preferred embodiment of the invention, the fragment according to the invention contains at least 10 nucleotides. In further embodiments, the fragment contains at least 15, in particular at least 25, preferably at least 50 and particularly preferably at least 100 nucleotides. The fragment according to the invention can therefore also be an oligonucleotide. The homology of the fragment according to the invention over its total length to the nucleic acid sequence shown in SEQ ID No. 1, SEQ ID No. 3 or SEQ ID No. 5 is at least 75%, in particular at least 80%, preferably at least 85%, particularly preferably at least 90%, and most preferably at least 95%, 96%, 97%, 98% or 99%.

Further embodiments of the invention relate to a vector which comprises at least one nucleic acid molecule according to the invention and/or at least one nucleic acid fragment under the functional control of at least one expression regulation element which guarantees the transcription of the nucleic acid into a translatable RNA and/or the translation of the RNA into a protein. The vector is preferably a plasmid, a cosmid, a virus, a bacteriophage, a shuttle vector or another vector customarily employed in genetic engineering. Suitable regulatory elements are, for example, promoters, enhancers, operators, silencers, ribosome binding sites and/or transcription termination signals, for example a 3′-transcription terminator. The regulatory elements which are connected functionally to the nucleic acid molecules according to the invention, for example the nucleic acid molecule having the nucleic acid sequence shown in SEQ ID No. 1, can originate from organisms or genes other than the nucleic acid molecules themselves. Of course, the regulation elements bonded functionally to the nucleic acid molecules according to the invention can also originate from Candida, in particular C. albicans. According to the invention, it is likewise proposed that the vector has a signal sequence for the transport of the expressed protein encoded by the nucleic acid molecule according to the invention into a cell organelle, a cell compartment, into the extracellular space or out of the cell. In a particularly preferred embodiment of the present invention, the nucleic acid molecules according to the invention can be present in antisense orientation to the regulatory element(s) connected functionally thereto such that an expression of an antisense mRNA is made possible which can inhibit or reduce the expression of the endogenous C. albicans RBR1 gene, the endogenous C. albicans RBR2 gene or the endogenous C. albicans RBR3 gene in a target organ. When using a fragment of the nucleic acid molecules according to the invention, this must have a length which suffices in order to make possible hybridization and inhibition of translation, for example a length of at least 10 nucleotides, in particular at least 15, at least 25, preferably at least 50 and particularly preferably at least 100 nucleotides.

The vectors according to the invention can additionally have further functional units which, for example, cause a stabilization and/or replication of the vector in a host organism or at least contribute thereto. The vectors according to the invention can, of course, also contain elements such as antibiotic resistance genes, selection markers or nucleic acid areas which encode affinity epitopes such as HA, Myc, maltose-binding protein, His6 or fluorescent proteins such as luciferase or Gfp.

The present invention likewise relates to a host cell which contains one or more of the nucleic acid molecules according to the invention and/or one or more of the above-mentioned vectors and in a preferred embodiment is able to express the cell wall protein Rbr1p, Rbr2p and/or Rbr3p according to the invention. The present invention likewise also relates to those cells which originate from a host cell which has been transformed using the above-mentioned nucleic acids and/or vectors according to the invention. Preferably, the host cells according to the invention are prokaryotic or eukaryotic cells such as bacterial, yeast, plant, insect or mammalian cells, in particular also human cells. A host cell according to the invention can comprise the inserted nucleic acid molecule according to the invention or the nucleic acid molecule contained in the vector heterologously with respect to the transformed cell. That is to say that the nucleic acid molecule which encodes a cell wall protein from Candida naturally does not occur in this cell or else is located at another site or in another copy number or orientation in the genome of the host cell than the corresponding naturally occurring nucleic acid. The nucleic acid molecule introduced according to the invention can, however, also be homologous with respect to the transformed cell.

In a preferred embodiment of the invention, the host cell according to the invention is a gram-negative prokaryotic cell, particularly preferably an enterobacteria cell. Particularly preferably, the host cell according to the invention is an Escherichia coli cell. In a further embodiment of the present invention, the host cell according to the invention can also be a eukaryotic cell, in particular a fungal cell, for example a cell of the genus Candida, preferably C. albicans, a Saccharomyces cell or an animal cell, in particular an insect or mammalian cell.

Particularly preferred examples of suitable C. albicans host cells are derivatives of the strain SC5314 (Fonzi and Irwin, Genetics, 134 (1993), 717-728). According to the invention, it is also provided that the host cell according to the invention is present in the form of a cell culture.

The technical problem underlying the present invention is also solved by the provision of a method for the production of a cell wall protein necessary for the hyphae development of a pathogenic fungal organism, in particular of the Rbr1p protein, of the Rbr2p protein and of the Rbr3p protein of Candida albicans, comprising the culturing of a host cell according to the invention in a suitable culture medium under conditions which allow the expression of the cell wall protein, and the obtainment of the expressed cell wall protein from the cell or from the medium. Numerous methods for the culturing of prokaryotic and eukaryotic host cells in culture medium, and the culturing conditions which lead to the expression of proteins, are known to the person skilled in the art. The person skilled in the art likewise knows numerous methods for the isolation of expressed protein from a cell and/or from the culture medium.

The present invention likewise relates to a protein which has the amino acid sequence shown in SEQ ID No. 2 and is encoded by the nucleic acid sequence shown in SEQ ID No. 1. In a preferred embodiment, the protein is the Rbr1p protein from C. albicans, where the protein according to the invention can be produced using the method according to the invention for the production of the cell wall protein necessary for hyphae development.

The invention also relates to a protein which has the amino acid sequence shown in SEQ ID No. 4 and which is encoded by the nucleic acid sequence shown in SEQ ID No. 3. In a preferred embodiment, the protein is the Rbr2p protein of C. albicans, where the protein according to the invention can be produced using the method according to the invention for the production of the cell wall protein necessary for hyphae development.

The invention further relates to a protein which has at least the amino acid sequence shown in SEQ ID No. 6 and which is encoded by the nucleic acid sequence shown in SEQ ID No. 5. In a preferred embodiment, the protein is the Rb3p protein of C. albicans, where the protein according to the invention can be produced using the method according to the invention for the production of the cell wall protein necessary for hyphoe development.

The present invention likewise relates to antibodies which specifically recognize a cell wall protein according to the invention, in particular the inventive cell wall protein Rbr1p, Rbr2p or Rbr3p of C. albicans, and bind thereto. In connection with the present invention, an “antibody” is understood as meaning a polypeptide which is essentially encoded by an immunoglobulin gene or a number of immunoglobulin genes, or fragments thereof which specifically binds/bind and recognizes/recognize an antigen. Known immunoglobulin genes include both the kappa, lambda, alpha, gamma, delta, epsilon and mu genes for the constant region and the numerous genes for the variable immunoglobulin region. Antibodies can be present, for example, as intact immunoglobulins or as a number of well characterized fragments thereof which are produced by means of cleavage with various peptidases. The term “antibody” used in the present description therefore comprises both intact antibody molecules and fragments thereof. The antibody fragments are in particular fragments such as Fab, F(ab′)₂ and Fv, which can bind the epitope determinants. The fragments can either have been produced by means of the modification of complete, whole antibodies or by means of de novo synthesis using DNA recombination techniques. The term “antibody” also includes modified antibodies, for example oligomeric, reduced, oxidized and/or labeled antibodies. The antibody can be both a monoclonal and polyclonal antibody. Methods for the production of monoclonal and polyclonal antibodies are known in the prior art.

An antibody according to the invention directed against the Rbr1p, Rbr2p or Rbr3p cell wall protein according to the invention therefore specifically recognizes the Rbr1p, Rbr2p or Rbr3p protein and binds specifically thereto. Preferably, the antibody or antibody fragments according to the invention do not bind any other antigens, for example other proteins present on the cell wall of Candida. In a preferred embodiment, an antibody according to the invention directed against the cell wall protein Rbr1p, Rbr2p or Rbr3p protein is able to recognize specifically only the cell wall protein Rbr1p protein or the Rbr2p cell wall protein or the Rbr3p cell wall protein of a Candida species and to bind thereto. That is to say, an antibody directed against the Rbr1p, Rbr2p or Rbr3p protein of C. albicans recognizes and binds exclusively the Rbr1p, Rbr2p or Rbr3p protein of C. albicans, but not the corresponding Rbr1p protein, the corresponding Rbr2p protein or the corresponding Rbr3p protein of a related Candida species, for example the Rbr1p protein, Rbr2p or Rbr3p protein of C. glabrata. Conversely, an antibody directed against the Rbr1p, Rbr2p or Rbr3p protein of C. glabrata does not recognize and bind the corresponding protein of C. albicans.

Of course, the antibodies or fragments according to the invention can be modified, for example conjugated, associated or covalently or noncovalently bonded with other molecules or other parts thereof, for example with a dye label, a radiolabel, an enzyme inducing a measurable reaction, such as a phosphatase or peroxidase, enzyme substrates, fluorescent substances, chemiluminescent substances, cytotoxic agents, spacers, vehicles or the like. The labeled, conjugated or unmodified antibodies can be present in soluble or immobilized form, for example on carrier matrices or beads such as nanoparticles.

The present invention of course also relates to an antibody which specifically recognizes an antibody directed against the cell wall protein Rbr1p and/or an antibody directed against the cell wall protein Rbr2p and/or an antibody directed against the cell wall protein Rbr3p and binds thereto.

The present invention also relates to a method for the characterization and/or for the detection of the hyphae stage of Candida cells or cells of pathogenic fungal organisms which are related to Candida, comprising the incubation of the cells or cell fractions thereof with an agent for the identification of the cell wall protein Rbr1p, Rbr2p, Rbr3p, a homologous protein thereof and/or of a fragment thereof, of the detection of the protein or of a fragment thereof indicating the presence of the virulent hyphae stage of the cells.

The present invention thus also solves the technical problem underlying it by the provision of protein markers, namely the cell wall protein Rbr1p, Rbr2p and Rbr3p of Candida, with the aid of which it is possible to determine unequivocally and exactly the hyphae stage of Candida cells. Since Rbr1p, Rbr2p and Rbr3p only occur in the cell wall of hyphally growing Candida cells, the three proteins are ideal markers with the aid of which the hyphal differentiation stage of Candida cells, for example C. albicans cells, can be unequivocally and rapidly detected and thus a clear distinction can be made between yeast cells and hyphae cells. The analysis and determination of the hyphal development stage of Candida using the Rbr1p, Rbr2p and Rbr3p proteins according to the invention thus also yields important information on the virulence of the fungus.

In connection with the present invention, the term “immunological agent” is in particular understood as meaning an antiserum directed against the cell wall protein Rbr1p, Rbr2p or Rbr3p, an antibody directed against the cell wall protein Rbr1p, Rbr2p or Rbr3p or a fragment thereof or a complex thereof. An “antiserum” is understood as meaning a serum which contains antibody against a monospecific antigen or a number of antigens or epitopes. The antiserum is obtained from animals or humans specifically immunized for this purpose, which/who have suffered a certain complaint, in particular a complaint caused by Candida species. On account of the heterogeneity of the immune response, the antiserum directed against an antigen, for example the antiserum directed against Rbr1p, Rbr2p or Rbr3p, contains different antibodies, “polyclonal antibodies”, which are produced by different plasma cell clones. According to the invention, it is proposed that the detection of Rbr1p, Rbr2p or Rbr3p or a fragment thereof by the immunological agent is carried out by means of a fluorescence method, a chemiluminescence method, an immunological method or a radiometric method. The method for the detection of the presence of the cell wall protein Rbr1p, Rbr2p or Rbr3p can optionally be carried out in combination with a microscopic method and an image analysis method.

In a preferred embodiment of the method according to the invention for the characterization and/or for the detection of the hyphae stage of Candida cells, the cell wall protein Rbr1p is the cell wall protein Rbr1p from C. albicans, which is encoded by a nucleic acid having the nucleic acid sequence shown in SEQ ID No. 1 and has the amino acid sequence shown in SEQ ID No. 2. In a further preferred embodiment of the method according to the invention for the characterization and/or for the detection of the hyphae stage of Candida cells, the cell wall protein Rbr2p is the cell wall protein Rbr2p from C. albicans, which is encoded by a nucleic acid having the nucleic acid sequence shown in SEQ ID No. 3 and has the amino acid sequence shown in SEQ ID No. 4. In a still further preferred embodiment of the method according to the invention for the characterization and/or for the detection of the hyphae stage of Candida cells, the cell wall protein Rbr3p is the cell wall protein Rbr3p from C. albicans, which is encoded by a nucleic acid having the nucleic acid sequence shown in SEQ ID No. 5 and has the amino acid sequence shown in SEQ ID No. 6.

In further embodiments of the method according to the invention, the cell wall protein Rbr1p, Rbr2p or Rbr3p is the Rbr1p, Rbr2p or Rbr3p protein or a homologous protein thereto from C. tropicalis, C. krusei, C. parapsilosis, C. guilliermondii, C. glabrata, C. dubliniensis, C. lusitaniae, a Trichosporon species or a Blastoschizomyces species, the detection of the protein preferably being carried out using an antibody which is directed specifically against the Rbr1p cell wall protein, the Rbr2p cell wall protein or the Rbr3p cell wall protein of C. tropicalis, C. krusei, C. parapsilosis, C. guilliermondii, C. glabrata, C. dubliniensis, C. lusitaniae, Trichosporon or Blastoschizomyces. According to the invention, the hyphal differentiation stage of C. tropicalis, C. krusei, C. parapsilosis, C. guilliermondii, C. glabrata, C. dubliniensis, C. lusitaniae, Trichosporon or Blastoschizomyces is characterized and/or detected thereby. Antibodies against the Rbr1p cell wall protein, the Rbr2p cell wall protein or the Rbr3p cell wall protein from one of the Candida species mentioned can, for example, be produced by isolating the Rbr1p cell wall protein, the Rbr2p cell wall protein or the Rbr3p cell wall protein from cells of the corresponding Candida species and is then, using methods known by the person skilled in the art, employed for the production of a monoclonal or polyclonal antibody. The Rbr1p, cell wall protein, the Rbr2p cell wall protein or the Rbr3p cell wall protein of a Candida species can also be obtained using one of the nucleic acid sequences shown in SEQ ID No. 1, SEQ ID No. 3 or SEQ ID No. 5 by employing this for the isolation of a homologous nucleic acid from cells of the corresponding Candida species. After isolation and sequencing of the homologous nucleic acid, the amino acid sequence of the Rbr1p cell wall protein encoded by the nucleic acid, the amino acid sequence of the Rbr2p cell wall protein encoded by the nucleic acid or the amino acid sequence of the Rbr3p cell wall protein encoded by the nucleic acid can then be derived on the basis of the nucleic acid sequence obtained. The corresponding protein can then, for example, be chemically synthesized in vitro and employed for the production of an antibody specifically directed against it.

According to the invention, it is proposed that the Candida cells to be characterized are present in a sample, preferably a biological sample. In connection with the present invention, a “biological sample” is in particular understood as meaning a skin or mucous membrane swab, an organ biopsy, a tissue biopsy, a body fluid, a body secretion, stool or a rinsing fluid which was obtained by rinsing cavities or hollow organs of a human or animal body. The sample can be taken both from a living or dead organism, organ or tissue. A “biopsy” is a tissue sample obtained by puncture with a hollow needle, using a special instrument such as a punch instrument, a biopsy probe or forceps or surgically using a scalpel. A “body fluid” is in particular sputum, urine, plural effusion, spinal fluid, lymph or blood. According to the invention, the blood can preferably be both an unpurified and a purified blood sample, blood plasma or blood serum. “Secretions” are secretions from cells, in particular glands.

According to the invention, a “biological sample” can also be a culture, for example a blood culture, that is a bacterial culture from a blood sample, or a culture medium, for example a fermentation medium, in which Candida cells or human, animal or plant cells have been cultured. A sample in the sense of the invention can also be an aqueous solution, emulsion, dispersion or suspension, which contains isolated and purified Candida cells or constituents thereof. A biological sample can already have been subjected to purification steps in order to isolate or enrich Candida cells but can also be present unpurified.

In a preferred embodiment of the method according to the invention, the Candida cells to be characterized which are employed are cells which have been isolated from a biological sample and enriched and are present as a cell preparation, preferably in a suitable buffer system. The Candida cells are preferably intact cells.

According to the invention, it is likewise proposed that, for the characterization and/or for the detection of the hyphal differentiation stage of Candida cells, cell fractions are also employed which are obtained by disruption and fractionation of Candida cells, isolated and preferably purified cell fractions being obtained. These cell fractions obtained in this way can then be employed in the method according to the invention for the characterization and/or for the detection of the hyphae growth stage of Candida cells. Methods for cell disruption and for the fractionation of cell constituents are well known in the specialty. Preferably, the cell fractions employed for characterization of the differentiation stages of Candida cells comprise at least one cell wall fraction of the Candida cells.

The technical problem underlying the present invention is also solved by a method for the detection of a Candida infection in a biological sample obtained from a human or animal organism, the presence of the protein Rbr1p, Rbr2p, Rbr3p, of a homologous protein thereof and/or of a fragment thereof in the biological sample and/or in the cell wall of Candida cells or cells of pathogenic organisms related to Candida optionally contained in the biological sample being detected, comprising
a) the incubation of the biological sample with an agent for the identification of the protein Rbr1p, Rbr2p, Rbr3p, a homologous protein thereof and/or a fragment thereof and
b) the detection of the interaction of the identification means with the protein or fragment thereof.

The method according to the invention for the detection of a Candida infection is thus based either on the direct detection of the Rbr1p, Rbr2p or Rbr3p cell wall protein of Candida present free in the sample or of a part thereof or else on the demonstration that the cell wall protein Rbr1p, Rbr2p or Rbr3p is present in the cell wall of Candida cells which are contained in the sample. In both cases, the detection of Rbr1p, Rbr2p and/or Rbr3p points to the occurrence of virulent hyphal Candida cells and thus to the presence of a Candida infection. The method according to the invention thus advantageously allows a rapid and exact detection of a Candida infection, in particular of an invasive candidiasis. The method according to the invention thus in particular makes possible a very early diagnosis of mycosis.

The direct detection according to the invention of the Candida cell wall protein Rbr1p, Rbr2p and/or Rbr3p in a sample or in the cell wall of Candida cells contained in the sample, in particular the immunological detection of the cell wall antigen by means of an antibody directed against the cell wall protein Rbr1p, the cell wall protein Rbr2p or the cell wall protein Rbr3p, has a considerably higher diagnostic value compared with the diagnosis methods known at present. Thus the cultural and/or histological detection of Candida carried out at present in most cases only makes possible the detection of a Candida infection in the late stage, while early stages frequently escape from this detection, in particular in immunosuppressed patients. The antibody titer against Candida antigens determined at present is also barely or not suitable at all up to now for the diagnosis of a Candida infection. The causes of this are varied. Thus it is known that healthy persons have antibodies against Candida antigens. On the other hand, some patients, in particular immunosuppressed patients, very often develop no adequate immune reaction in the case of a Candida infection. A further cause consists in the fact that often serum samples are taken before antibodies have formed.

In connection with the present invention, a “Candida infection” is understood as meaning a “Candida mycosis” or candidosis or candidiasis, that is an infection, disease or a disease state which is caused by a Candida species. In particular, it concerns those complaints which are caused exclusively by Candida species. The term “Candida infection” comprises all complaints which are caused by Candida species such as C. albicans, C. tropicalis, C. krusei, C. parapsilosis, C. guilliermondii, C. glabrata, C. dubliniensis, C. lusitaniae, a Trichosporon species or a Blastoschizomyces species. In context with the invention, the term “Candida infections” is also understood as meaning diseases or disease states which primarily have other causes and in which the above-mentioned Candida species are only involved in the overall clinical picture or add additional symptoms, for example opportunistic infections.

An invasive “candidiasis” is understood according to the invention as meaning Candida infections in the blood circulation and in internal organs. Invasive candidiasis in particular occurs when cells of a Candida species reach the blood circulation, lead to an infection there and are dispersed through the entire body from there. The most frequent symptoms of invasive candidiasis include fever and shivering fits. If the infection spreads to deeper lying organs such as the kidneys, liver or eyes, additional specific symptoms can develop.

According to the invention, it is provided that an immunological agent is employed for the identification of Rbr1p, Rbr2p or Rbr3p as a cell surface antigen of Candida cells or else as a free antigen, in particular circulating in body fluids, in particular an antiserum directed against Rbr1p, Rbr2p or Rbr3p, an antibody directed against Rbr1p, Rbr2p or Rbr3p or a fragment thereof or a complex thereof. The antibody employed according to the invention can be both a monoclonal and a polyclonal antibody, which preferably has a label, in particular a dye label, a radiolabel, a fluorescent label, a chemiluminescent label or an enzyme inducing a measurable reaction. The investigation of the interaction of the identification means, preferably of the antibody, with the Rbr1p cell wall antigen, Rbr2p cell wall antigen or Rbr3p cell wall antigen is carried out according to the invention by means of an immunological method, a fluorescence method, a chemiluminescence method or a radiometric method, optionally in combination with a microscopic method and/or an image analysis method.

In a preferred embodiment, the protein to be detected is the cell wall protein Rbr1p of C. albicans, which is encoded by a nucleic acid molecule having the nucleic acid sequence shown in SEQ ID No. 1 and which contains the amino acid sequence shown in SEQ ID No. 2. In a further preferred embodiment, the protein to be detected is the cell wall protein Rbr2p of C. albicans, which is encoded by a nucleic acid molecule having the amino acid sequence shown in SEQ ID No. 3 and which contains the amino acid sequence shown in SEQ ID No. 4. In a still further preferred embodiment, the protein to be detected is the cell wall protein Rbr3p of Candida albicans, which is encoded by a nucleic acid molecule having the amino acid sequence shown in SEQ ID No. 5 and which contains the amino acid sequence shown in SEQ ID No. 6. By means of the detection of Rbr1p, Rbr2p and/or Rbr3p of C. albicans in the sample or the detection of C. albicans-Rbr1p, C. albicans-Rbr2p and/or C. albicans-Rbr3p in the cell wall of Candida cells which are contained in the biological sample, according to the invention invasive fungal complaints can be detected which are caused in particular by C. albicans, in particular an invasive candidiasis caused by C. albicans. By means of the detection of the Rbr1p protein, Rbr2p protein or Rbr3p protein of C. tropicalis, C. krusei, C. parapsilosis, C. guilliermondii, C. glabrata, C. dubliniensis, C. lusitaniae a Trichosporon species or a Blastoschizomyces species, according to the invention invasive fungal complaints can be detected which are caused in particular by C. tropicalis, C. krusei, C. parapsilosis, C. guilliermondii, C. glabrata, C. dubliniensis, C. lusitaniae, a Trichosporon species or a Blastoschizomyces species, in particular an invasive candidiasis caused by one of the Candida species mentioned.

According to the invention, it is further provided that the detection of the Candida infection can be carried out using a biological sample which can contain Candida cells, but does not have to contain them. The biological sample can be, for example, a skin or mucous membrane swab, an organ biopsy, a tissue biopsy, a body fluid, a body secretion, stool or a rinsing fluid.

In a preferred embodiment of the invention, the detection of the Candida infection is carried out using a body fluid which, in comparison with other samples such as a biopsy, has the advantage that the probability is very high that Rbr1p, Rbr2p and/or Rbr3p are circulating freely. The body fluid is in particular a blood sample which can be taken directly from the body and can be employed in the method according to the invention without further purification. The method according to the invention can also be carried out using a blood culture. Likewise, according to the invention blood products such as blood plasma or blood serum can be employed. The detection of the cell wall protein Rbr1p, Rbr2p and/or Rbr3p circulating freely in the blood or the demonstration that the Candida cells contained in the blood have Rbr1p, Rbr2p and/or Rbr3p on their cell surface, an invasive candidiasis in the blood can be detected. That agent employed for the identification of Rbr1p, Rbr2p and/or Rbr3p is preferably an immunological agent, in particular an antiserum directed against Rbr1p, Rbr2p or Rbr3p, an antibody directed against Rbr1p, Rbr2p or Rbr3p or a fragment thereof or a complex thereof. In the case of the use of an antibody, this can have a label, selected from the group consisting of a dye label, a radiolabel, a fluorescent label, a chemiluminescent label or an enzyme inducing a measurable reaction.

The present invention likewise relates to a method for the discovery and identification of substances having therapeutic action against diseases which are caused by Candida species or pathogenic fungal species which are related to Candida. According to the invention, it is provided here that a substance to be tested is brought into contact in a suitable medium with at least one agent, selected from the group consisting of a nucleic acid molecule according to the invention, a nucleic acid fragment according to the invention, a vector according to the invention, a host cell according to the invention, a cell wall protein according to the invention or an antibody directed against this cell wall protein and an interaction between the substance to be tested and the agent is detected.

For example, the nucleic acid molecules according to the invention, the fragments of these nucleic acid molecules according to the invention, the proteins according to the invention or the antibodies according to the invention are immobilized on appropriate chips. These chips can then subsequently be brought into contact in a suitable medium with the substance to be tested for the detection of an interaction between a substance to be tested and the agent immobilized on the chips. In this manner, for example, the nucleic acid molecules, proteins or antibodies according to the invention can be used in order to identify substances, for example proteins, which in vivo themselves bind to nucleotide sequences which encode hyphae-specifically expressed cell wall proteins such as Rbr1p, Rbr2p or Rbr3p or regulate the expression of these proteins, or to the hyphae-specifically expressed cell wall proteins. Such binding substances, in particular proteins, can potentially be employed as medicaments against Candida-caused diseases if, for example, they are able to inhibit or to suppress the transcription of the hyphae-specific cell wall proteins according to the invention by binding to regulatory nucleotide sequences or if by binding to the inventive hyphae-specifically expressed cell wall proteins they can inhibit or suppress their activity. If such substances which bind to nucleic acid molecules or proteins according to the invention induce or promote the transcription of the hyphae-specific cell wall proteins according to the invention or favor the activity of the hyphae-specifically expressed cell wall proteins according to the invention, these substances can potentially be used as medicaments for the treatment of Candida-caused diseases.

The present invention likewise relates to a diagnostic composition, comprising at least one nucleic acid molecule according to the invention, at least one nucleic acid fragment according to the invention, at least one vector according to the invention, at least one host cell according to the invention, at least one protein according to the invention and/or at least one antibody according to the invention.

A further preferred embodiment of the invention relates to a pharmaceutical composition, comprising at least one nucleic acid molecule according to the invention, at least one nucleic acid fragment according to the invention, at least one vector according to the invention, at least one host cell according to the invention, at least one protein according to the invention, at least one antibody according to the invention and/or at least one substance whose therapeutic activity has been detected by means of the method according to the invention, for the identification of substances having therapeutic action.

The nucleic acid contained in the diagnostic or pharmaceutical composition according to the invention can thus be a protein-encoding nucleic acid, in particular a nucleic acid which encodes the cell wall protein Rbr1p according to the invention of C. albicans, the cell wall protein Rbr2p according to the invention of C. albicans or the cell wall protein Rbr3p according to the invention of C. albicans. The nucleic acid contained in the diagnostic or pharmaceutical composition can, however, also contain a nucleic acid sequence which encodes the Rbr1p, Rbr2p or Rbr3p of C. tropicalis, C. krusei, C. parapsilosis, C. guilliermondii, C. glabrata, C. dubliniensis, C. lusitaniae, a Trichosporon species or a Blastoschizomyces species. The nucleic acids which encode one of the proteins of one of the aforementioned Candida species have a very great homology over their entire length to a nucleic acid encoding C. albicans-Rbr1p, C. albicans-Rbr2p or C. albicans-Rbr3p, the homology being more than 75%, in particular more than 80%, preferably more than 85%, particularly preferably more than 90% and most preferably more than 95% or more than 98%.

The cell wall protein Rbr1p, Rbr2p or Rbr3p contained in the diagnostic or pharmaceutical composition according to the invention can be present in the composition, for example, as a complex with other proteins. These proteins are preferably those which can likewise be employed for the diagnosis and/or therapy of Candida infections.

The protein contained in the diagnostic or pharmaceutical composition according to the invention can also be a fragment of a wild-type protein or a derivative of a wild-type protein. A “derivative” is understood as meaning a functional equivalent or a functional derivative of the wild-type protein which, with retention of the parent structure of Rbr1p, Rbr2p or Rbr3p, is obtained by substitution of atoms or molecule groups or molecule radicals and/or whose amino acid sequence differs in at least one amino acid position of the wild-type protein.

The differences between the cell wall protein derivative and the wild-type cell wall protein can be based on naturally occurring or artificially produced mutations, for example by means of molecular biology techniques known to the specialty, in the nucleic acids encoding the derivatives. The differences can have also been produced by means of chemical methods, for example during the chemical synthesis of the protein derivatives. Derivatives are also fusion proteins in which functional domains of another protein are contained on the N-terminus and/or C-terminus of Rbr1p, Rbr2p or Rbr3p. The derivatives of Rbr1p, Rbr2p or Rbr3p used according to the invention have a homology of at least 75%, preferably at least 80%, in particular at least 85%, preferably at least 90%, more preferably at least 95% and most preferably at least 96%, 97%, 98% or 99% to the corresponding wild-type cell wall proteins, in particular to C. albicans Rbr1p having the amino acid sequence shown in SEQ ID No. 2, to C. albicans Rbr2p having the amino acid sequence shown in SEQ ID No. 4 or to C. albicans Rbr3p having the amino acid sequence shown in SEQ ID No. 6.

According to the invention, the protein derivatives contained in the diagnostic or pharmaceutical composition can have a modified activity, a modified stability, a modified specificity, a modified temperature, a modified pH and/or concentration profile and/or a modified effector pattern compared with the wild-type protein. The cell wall protein derivative used according to the invention can occur in other conformations and also contain other subunits or pre- and/or post-translational modifications.

According to the invention, it is also provided that the diagnostic or pharmaceutical compositions can contain an antibody which specifically recognizes the cell wall protein Rbr1p, the cell wall protein Rbr2p or the cell wall protein Rbr3p of C. albicans or of C. tropicalis, C. krusei, C. parapsilosis, C. guilliermondii, C. glabrata, C. dubliniensis, C. lusitaniae, a Trichosporon species or a Blastoschizomyces species and binds thereto and thus makes possible its detection. The antibody can be both a monoclonal and a polyclonal antibody. According to the invention, only one antibody fragment, for example Fab, F(ab′)₂ and Fv, can also be contained in the diagnostic or pharmaceutical composition.

In a further embodiment, the diagnostic or pharmaceutical composition can contain an antibody which specifically recognizes an antibody which is directed against Rbr1p of C. albicans, against Rbr2p of C. albicans or against Rbr3p of C. albicans and binds thereto and thus makes possible the detection of this antibody, for example in a biological sample such as blood.

In a particularly preferred embodiment, the pharmaceutical composition is a vaccine which contains at least one fungal cell wall protein, in particular the cell wall protein Rbr1p of C. albicans having the amino acid sequence shown in SEQ ID No. 2, the cell wall protein Rbr2p of C. albicans having the amino acid sequence shown in SEQ ID No. 4 and/or the cell wall protein Rbr3p of C. albicans having the amino acid sequence shown in SEQ ID No. 6 and which is suitable for the active immunization of a human or animal body against a Candida infection. In connection with the present invention, an “active immunization” is understood as meaning a parenteral administration of antigens which are not capable of proliferation, the aim consisting in forming in the human or animal body an antibody against the antigen and thus against the microorganism from which the antigen originates. With an active immunization, it can also be a local administration, in particular oral, nasal or cutaneous administration or inhalation, of microbial antigens not capable of proliferation, the aim consisting in building up a local infection defense on mucous membranes by formation of secretory antibodies and by increasing the macrophage activity.

In a further preferred embodiment, the pharmaceutical composition is a vaccine which contains at least one antibody selected from the group consisting of an antibody which specifically recognizes the Rbr1p of Candida and binds thereto, an antibody which specifically recognizes the Rbr2p of Candida and binds thereto, and an antibody which specifically recognizes the Rbr3p of Candida and binds thereto, and which is suitable for the passive immunization of a human or animal body against a Candida infection. In connection with the present invention, a “passive immunization” is understood as meaning the injection of immunoglobulin preparations, that is specific antibodies, or serum of an actively immunized human or animal, the aim consisting in transferring antiinfectious or antitoxic antibodies to a human or animal body for the prevention or treatment of infectious diseases.

According to the invention, it is provided that the vaccine is present as a lyophilizate or as an aqueous colloidal solution or suspension. The vaccine according to the invention can additionally contain at least one adjuvant.

The invention likewise relates to a kit for the identification of the cell wall protein Rbr1p of Candida species or of a pathogenic organism related to Candida and/or for the detection of the virulence of Candida cells or of cells of an organism which is related to Candida, comprising at least one container having an antibody which specifically recognizes the Rbr1p, Rbr2p or Rbr3p or a fragment thereof and binds thereto. In a preferred embodiment, the kit can be employed for in vitro identification of Rbr1p, Rbr2p or Rbr3p and/or for the in vitro detection of the virulence of Candida cells. The kit can optionally comprise a second container having the isolated and purified cell wall protein Rbr1p, Rbr2p and/or Rbr3p of C. albicans. The isolated and purified cell wall protein Rbr1p or Rbr2p or Rbr3p especially serves as a control.

The present invention likewise relates to the use of a nucleic acid molecule according to the invention, of a nucleic acid fragment according to the invention, of a vector according to the invention, of a host cell according to the invention, of a cell wall protein according to the invention or of an antibody according to the invention for the diagnosis of diseases of a human or animal organism which are caused by Candida species or pathogenic fungal organisms which are related to Candida.

The present invention likewise relates to the use of a nucleic acid molecule according to the invention, of a nucleic acid fragment according to the invention, of a vector according to the invention, of a host cell according to the invention, of a protein according to the invention or of an antibody according to the invention for the production of a diagnostic composition for the diagnosis of diseases of a human or animal organism which are caused by Candida species or pathogenic fungal organisms which are related to Candida.

In a further preferred embodiment, the present invention likewise relates to the use of an agent which decreases or inhibits the expression and/or of activity of Rbr1p, Rbr2p, Rbr3p and/or of a homologous protein thereof, as an active compound for the treatment and/or prevention of diseases of a human or animal organism which are caused by Candida species or pathogenic fungal organisms which are related to Candida. The present invention likewise relates to the use of an agent which decreases or inhibits the expression and/or activity of Rbr1p, Rbr2p, Rbr3p and/or of a homologous protein thereof, as an active compound for the production of a pharmaceutical composition for the treatment and/or prevention of diseases which are caused by Candida species or pathogenic fungal organisms related thereto. According to the invention, the agent is selected from the group consisting of the nucleic acid molecules according to the invention, the nucleic acid fragments according to the invention, the vectors according to the invention, the host cells according to the invention, the proteins according to the invention, the antibodies according to the invention or the substances in which, by means of the method according to the invention for the identification of therapeutically active substances, it has been demonstrated that they are suitable as a therapeutic for the treatment of diseases caused by Candida.

The present invention likewise relates to the use of a nucleic acid molecule according to the invention, of a nucleic acid fragment according to the invention, of a vector according to the invention, of a host cell according to the invention, of a protein according to the invention or of an antibody according to the invention for the identification and/or for the detection of substances which inhibit the expression or activity of Rbr1p, Rbr2p and/or Rbr3p in a pathogenic fungal organism and are suitable as an active compound for the production of a pharmaceutical composition.

In a further preferred embodiment, the present invention relates to the use of the nucleic acid molecule according to the invention having one of the nucleotide sequence shown in SEQ ID No. 1, SEQ ID No. 3 or SEQ ID No. 5, of a nucleic acid molecule having a nucleotide sequence which encodes a protein having one of the amino acid sequences shown in SEQ ID No. 2, SEQ ID No. 4 or SEQ ID No. 6, or of a fragment of one of these nucleic acid molecules for the isolation of a homologous nucleic acid which encodes the Rbr1p protein, the Rbr2p protein or the Rbr3p protein of C. tropicalis, C. krusei, C. parapsilosis, C. guilliermondii, C. glabrata, C. dubliniensis, C. lusitaniae, of a Trichosporon species, of a Blastoschizomyces species or of another fungal pathogenic organism.

The invention likewise relates to the use of an antibody according to the invention for the characterization and/or for the detection of the virulent hyphae stage of Candida cells.

The invention is explained in more detail by the following sequence listing, the following figures and following examples.

The sequence listing is part of the description and contains the sequences shown in SEQ ID No. 1 to 6.

SEQ ID No. 1 shows a nucleic acid sequence comprising 336 nucleotides, which encodes the amino acid sequence of the cell wall protein Rbr1p of Candida albicans shown in SEQ ID No. 2.

SEQ ID No. 2 shows the amino acid sequence comprising 111 amino acid residues of the cell wall protein Rbr1p of Candida albicans.

SEQ ID No. 3 shows a nucleic acid sequence comprising 507 nucleotides, which encodes the amino acid sequence of the cell wall protein Rbr2p of Candida albicans shown in SEQ ID No. 4.

SEQ ID No. 4 shows the amino acid sequence comprising 168 amino acid residues of the cell wall protein Rbr2p of Candida albicans.

SEQ ID No. 5 shows a nucleic acid sequence comprising 1682 nucleotides which encodes the amino acid sequence of the cell wall protein Rbr3p of Candida albicans, a part of the amino acid sequence being shown in SEQ ID No. 6.

SEQ ID No. 6 shows a subarea comprising 560 amino acid residues of the amino acid sequence of the cell wall protein Rbr3p of Candida albicans.

The figures show:

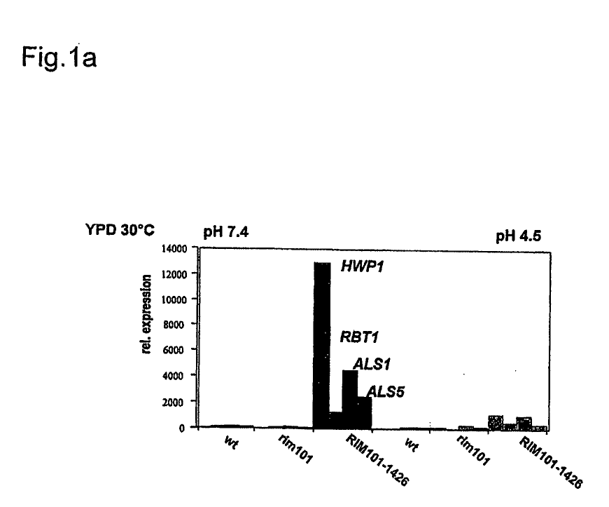

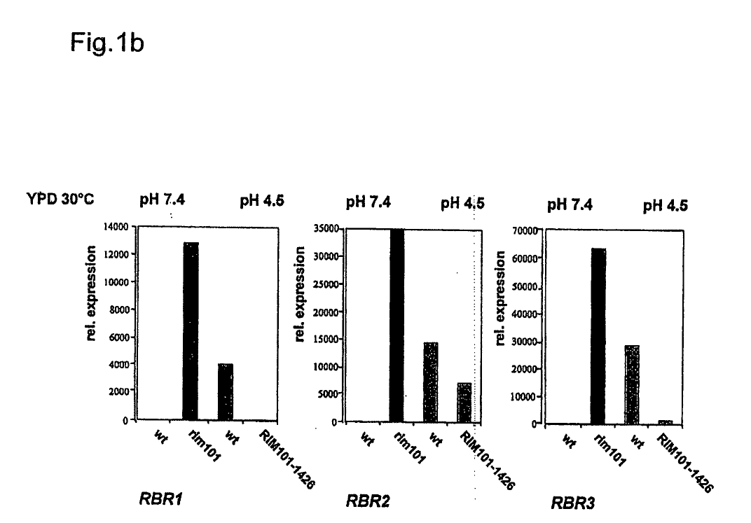

FIG. 1 shows that the transcription factor Rim101p activates or represses cell wall genes of C. albicans. 1a) Activation of hyphal genes by RIM101. 1b) Repression of RBR1, RBR2 and RBR3, by RIM101. RNA for the Northern blot analysis was obtained from the wild-type strain SC1453, the strain RIM101-1426 and Δrim101 mutant strain, which had in each case been cultured for 6 hours in YPD medium with 100 mM HEPES buffer at 30° C. In the case of SC1453 and of the mutant strain Δrim101 (dark bar) a pH of 7.4 was set. With SC1453 and RIM101-1426 (light bar) a pH of 4.5 was set. The quantitative determination of the mRNA concentration was carried out by standardization of the hybridization signals against the ACT1 signal of SC5314 at a pH of 7.4.

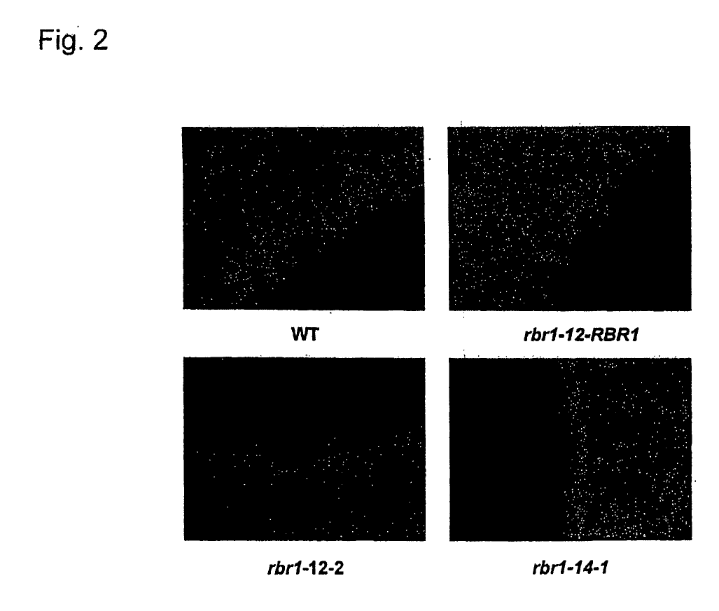

FIG. 2 shows the pH-dependent filamentation defect of Δrbr1 mutants on M-199 soft agar. The strains were pre-cultured in SC-ura medium for 24 hours and pelleted by means of a short centrifugation. 5 μl of concentrated cell suspensions were applied to the agar surface. The plates were incubated at 30° C. for 72 h.

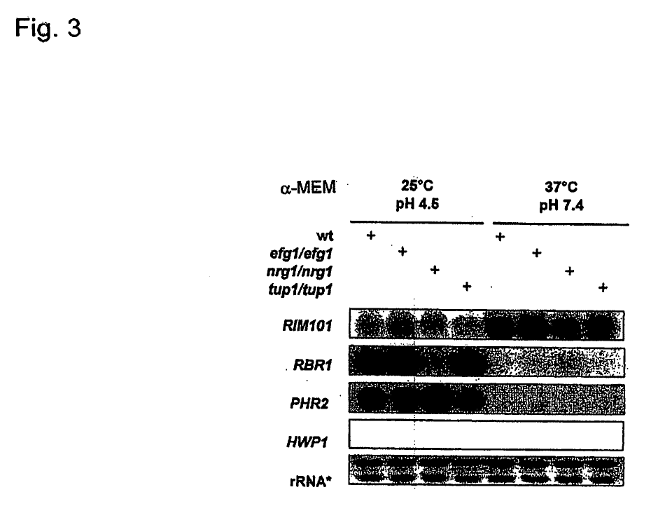

FIG. 3 shows that the RBR1 expression is activated by NRG1. The transcript quantities of RBR1 in wild-type, Δefg1, Δnrg1 and Δtup1 mutant strains were determined by means of Northern blotting. RNA which was obtained from strains which had been cultured in α-MEM medium for 4 h at a pH of 4.5 (running tracks 1-4) or a pH of 7.4 (running tracks 5-8) at non-induced temperatures of 25° C. or at hyphae-induced temperatures of 37° C. rRNA*: ethidium bromide-stained control.

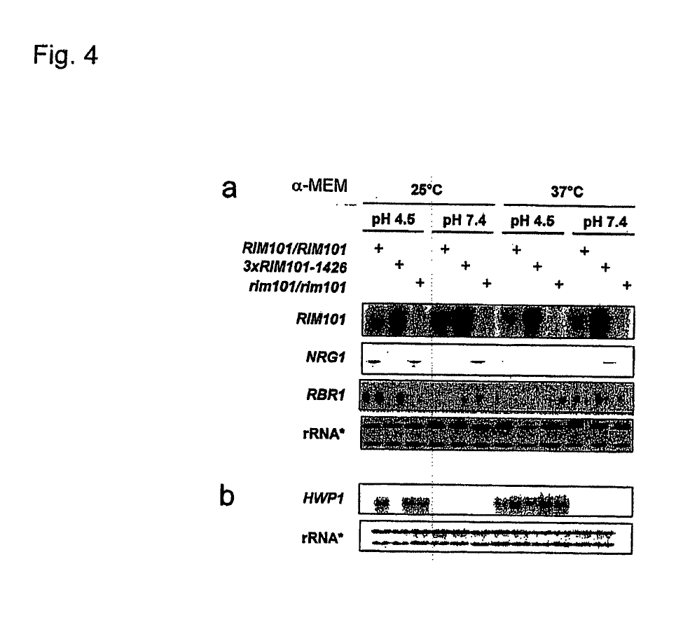

FIG. 4 (a) shows that the expression of RBR1 and NRG1 is dependent on RIM101 and on the temperature. The strains SC1453, RIM101-1426 and Δrim101 were cultured for 6 h in α-MEM medium, 100 mM HEPES buffer at a pH of 4.5 and a pH of 7.4 at 25° C. and 37° C. in order to isolate RNA for the Northern blot analysis. rRNA*: ethidium bromide-stained control.

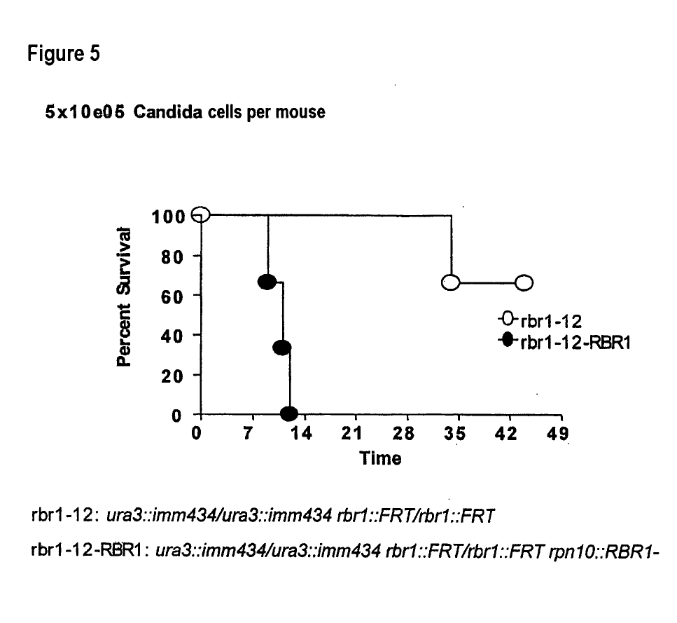

FIG. 5 shows the results which were obtained using different Candida strains in a mouse model for systemic candidosis. The results show that Rbr1p is necessary for the virulence of Candida and for a successful infection of the host by Candida.

### EXAMPLE

Materials and Methods

C. albicans Strains

The C. albicans strains used are listed in table 1. The DNA microarray experiments were carried out using three different strains:

a) the wild-type strain (SC5314),

b) the homozygous deletion strain Δrim101 (CAF3- ×ura3::imm434/ura3::imm434 rim101::hisG/rim101::hisG::URA3) (El Barkani et al., Mol. Cell. Biol., 20 (2000), 4635-4647) and

c) the strain RIM101-1426 (CAF3-16-2 ura3::imm434/ura3::imm434 RIM101/RIM101-(pBSK+-RIM101-1426-URA3)_{n>2}) which overexpresses the dominant active protein RIM101-1426p (El Barkani et al., 2000).

The RBR1 deletion mutants were constructed by stepwise homologous recombination of CA14 using a URA3 flipper cassette (Morschhauser et al., Mol. Microbiol., 32 (1999), 547-556). An internal and an external pair of sequences, which flank the coding sequence of RBR1, were amplified by means of PCR. The external pair of the flanking regions (FR1 and FR2) was used for the deletion of the first allele. The internal flanking regions (FR3 and FR4) were used for the deletion of the second RBR1 allele. The following primer pairs were used for the amplification of the external and internal flanking regions by means of PCR in a Peltier thermal cycler 200 device (MJ Research) with 30 cycles at 55° C.:

| | |
|---|---|
| 1) | |
| FR1.for: | 5′-AAGGGCCCCACAAAATAAAAGCAGCAGGAA |
| and | |
| FR1.rev: | 5′-CCGCTCGAGTTCCAACTTTAATCCCGCAC |
| | (product length 457 bp); |
| 2) | |
| FR2.for: | 5′-ATAAGAATGCGGCCGCTTGCCACCAGTCAAATTCAA |
| and | |
| FR2 rev: | 5′-CGAGCTCCCGAAATGCCACCATAGTTT |
| | (product length 527 bp); |
| 3) | |
| FR3.for: | 5′-AAGGGCCCGTGCGGGATTAAAGTTGGAA |
| and | |
| FR3.rev: | 5′-CCGCTCGAGTTGTTGTTGTAAGCGAAGCC |
| | (product length 563 bp); |
| 4) | |
| FR4.for: | 5′-ATAAGAATGCGGCCGCTGAATGAGAATGAGGGGGAC |
| and | |
| FR4.rev: | 5′-CGAGCTCTTGAATTTGACTGGTGGCAA |
| | (product length 565 bp). |

The primer sequences contained unique cleavage sites (underscored in the sequences) in order to make possible direct ligation of the flanking regions in the plasmid pSFU1 (Morschhauser et al., 1999). FR1 and FR3 were to be ligated into the vector after cleavage using the restrictases ApaI and XhoI. FR2 and FR4 were inserted after cleavage using NotI and SacI, the plasmids pSFUR1-2 for the construct with the external deletion and pSFUR1-4 for the construct with the internal deletion being obtained. For the reversion of the homozygous Δrbr1 strain, the encoding sequence and the promoter region of RBR1 were cloned into the integrative C. albicans expression vector pCaExp (Care et al., Mol. Microbiol., 34 (1999), 792-798), the primers:

| | |
|---|---|
| 2736f-1.FR1.for: | AAGGGCCCCACAAAATAAAAGCAGCAGGAA |
| and | |
| RBR1.RVT.rev: | CCGCTCGAGCCGAAATGCCACCATAGTTT |

being used.

The vector contained the URA3 gene under the control of the native promoter and was designed for integration into the RP10 locus.

Three independent Δrbr1 mutant strains were transformed using pCaExp or pCaExp-RBR1. Transformants which express URA3 were selected on synthetic complete medium without uridine (SC-uri). Individual colonies were removed and cultured at 30° C. for 6 h in SC-uri medium and subsequently the recombination of the RBR1 locus was confirmed by means of Southern blot analysis. For the determination of the mRNA concentration by means of Northern blot experiments, the strains were cultured at 30° C. in A-MEM medium (pH 4.5).

Media and Growth Conditions

All media used contained final concentrations of 100 mM HEPES buffer and 0.1 mM uridine. The desired pHs were set either using 1 M NaOH (pH 7.4) or 1 M HCl (pH 4.5) before sterile filtration. For DNA microarray experiments, cells from cultures which had been incubated overnight at 30° C. in YPD medium were pelleted, decanted and resuspended in the residual medium. Prewarmed media having an identical pH were inoculated with the cell suspensions up to an optical density OD₆₀₀=0.05. The Δrim101 mutants and the wild-type control strain were cultured at a pH of 4.5. In order to make possible a comparison with the RIM101-1426 mutants and the wild-type strain, both strains were also cultured at a pH of 7.4. In order to avoid undesired effects on account of significant changes in the pH, the pH was determined at the end of each experiment. The changes in the pH were not more than 0.2 pH units. If Northern blot experiments were carried out for the confirmation of the DNA microarray data, the strains were cultured under identical conditions.

Northern blot experiments and phenotypic investigations were carried out in YPD medium, α-MEM medium (GIBCO), containing 2% glucose, or tissue culture medium M-199 (GIBCO) containing 0.1 mM uridine and 101 mM HEPES. The strains were cultured at 25° C., 29° C., 30° C. or 37° C. at acidic and neutral pHs. Solid media contained either 2% agar or 0.3% agar for soft agar plates, a 4% strength heated agar solution being added to the previously warmed media.

C. albicans Cell Wall-Specific DNA Microarray

The cell wall-specific DNA microarray was described by Sohn et al. (Mol. Microbiol., 47 (2003), 89-102). The array contains 117 different probes for known genes and open reading frames of C. albicans which have still not been characterized up to now. 65 of these genes are homologs of cell wall protein-encoded genes of S. cerivisiae. The array further detects genes which encode proteins of which it is already known that these are located in the cell wall of C. albicans. For the control, probes for actin (ACT1 (X16377)) were included.

Probe Amplification and Production of DNA Microarrays

The probes were amplified by means of PCR of genomic DNA from C. albicans in a volume of 100 μl on microtiter plates having 96 hollows by means of a Peltier thermal cycler 200 device (MJ Research). The PCR was carried out in PCR buffer (100 mM tris HCl, pH 8.8, 600 mM KCl, 15 mM Mg₂Cl, containing 1 M betaine (Sigma), 0.3 μm Cresol Red (Sigma). One unit of Taq polymerase, 20 ng of template and in each case 0.25 μm of each primer and 0.2 mM of each dNTPs were added and subsequently 30 PCR cycles were carried out, the denaturation being carried out at 94° C., the addition reaction at 50° C. and the extension reaction at 72° C. for 1 minute in each case. The PCR products were analyzed by means of gel electrophoresis in 1% strength agarose gels. Before the probes were applied to polylysine-coated glass microscope slides, the liquid was evaporated overnight at 8° C., such that a final concentration of betaine of 1.5 to 2 M was obtained.

For the preparation of the DNA microarrays, glass microscope slides (J. Melvin Freed Brand, Sigma) were washed for 2 h in 2.5 M NaOH in 60% EtOH on a shaker. The microscope slides were rinsed five times in fresh ddH₂O and coated with polylysine by a one-hour incubation in 0.01% polylysine (Sigma) in 0.1×PBS. Afterward, the microscope slides were washed briefly in ddH₂O and dried by means of centrifugation, a 30-minute treatment at 50° C. subsequently being carried out. The PCR probes were pressed onto the coated microscope slides by means of a GMS417 microarray device (Genetic Microsystems). For aftertreatment, the arrays were rehydrated for 5-10 min by means of 2×SSC and dried at 80° C. for 2 s on an inverted heating block. The DNA was crosslinked by means of UV light of 65 mJ in a Stratalinker device (Stratagene). The microscope slides were incubated for 20 min in blocking solution (170 mM succinic anhydride, 70 mM sodium borate, pH 8.0, in 1-methyl-2-pyrrolidinone) and afterward washed for 2 min in boiling ddH₂O. After a brief rinse in 95% EtOH, the microscope slides were dried by means of centrifugation.

Isolation of RNA, Fluorescent Labeling of cDNA and Hybridization on the Microscope Slides

After a 6-hour cell culture, the optical density was determined and the cells were harvested by means of a brief centrifugation at 1700×g. Cell pellets obtained were immediately frozen in liquid nitrogen and the pH of the supernatant was determined. Total RNA was isolated by means of a method using acidic phenol: one volume of acidic phenol (Roth) and one volume of TES (10 mM tris HCl, pH 7.5, 10 mM EDTA, 0.5% SDS) were added to the frozen cell pellets and subsequently a 45-minute incubation at 65° C. was carried out with occasional vortex treatment. Subsequently, a further phenol extraction for 10 min at 65° C. and finally a chloroform extraction at room temperature were carried out. Afterward, the RNA was precipitated using 0.3 M NaAc in 70% EtOH. In the case of RNA which was employed for the cDNA synthesis, a precipitation in 2 M LiCl was routinely carried out.

Fluorescent-labeled cDNA for the microarray hybridization was obtained by reverse transcription of 30 μg of RNA using Superscript II (Invitrogen). Afterward, labeling was carried out using a standard labeling protocol, either cy3-dUTP or cy5-dUT5 (Amersham) being employed. Labeled cDNA was mixed with a control cDNA which was provided with a different label, washed with TE buffer, pH 7.4, purified and adjusted to a final volume of 12 to 15 μl in 4×SSC and 0.2% SDS using Microcon YM-30 filters (Millipore). A hybridization on the array was carried out under a cover glass at 63° C. overnight in a water bath. After hybridization, the cover glass and the unbound cDNA were removed by stepwise washing with 1×SSC/0.03% SDC, 0.2×SSC and 0.05×SSC. The chips were dried by means of centrifugation and immediately scanned.

Analysis of the DNA Microarray Data

The DNA microarrays were scanned by means of epi-fluorescence microscopy using a GMS 418 array scanner (Genetic Microsystems). For photoexcitation, the following wavelengths were employed: 532 nm for Cy3-labeled cDNA and 635 nm for Cy5-labeled cDNA. Fluorescent spot signals of 16-bit TIFF images in the Cy3 and Cy5 channels were superimposed and quantified using the ImaGene software version 3.0 (Bio Discovery, Los Angeles, USA). The spots were quantified as the mean value of all pixel values in the spot region. The background for each spot was quantified separately as a mean value of all pixel values background region. The background values were subtracted from the corresponding spot values, net signal values being obtained. The net signal values below “1” were assessed as “1”. Probes with net signal values below “1000” for both channels were not taken into consideration in the calculation under the corresponding conditions. A different transcription was indicated as a ratio of the signal values of both channels. The data are mean values of two values of at least three different experiments. The DNA microarray data were for the most part confirmed by means of Northern blot analysis under identical conditions (compare strains and growth conditions).

Northern Hybridization

15 μg of total RNA were separated by means of electrophoresis on denaturing gels (1% agarose, 0.02 M MOPS, 8 mM NaAc, 1 mM EDTA and 2.2 M formaldehyde). RNA was applied to Hybond-N (Amersham) nylon membranes using 20×SSC corresponding to standard protocols (Sambrook, 1989) and UV-crosslinked twice on the moist blots at 120 mJ in a Stratalinker device (Stratagene). The blots were either pre-hybridized for 1 h at 65° C. or frozen at −80° C. before they were further processed. The PCR-amplified probes were purified and subsequently labeled by means of a Klenow reaction using [α-³²P]dCTP and random primers (Stratagene). The hybridization with individual probes was carried out over a period of at least 6 h. Subsequently, three washing steps were carried out in 1% SSC, 0.1% SDS. The blots were developed over a period of 24 to 72 h using a phosphor screen (Molecular Dynamics). The screens were scanned using a phosphor imager device (Molecular Dynamics). Individual bands were detected quantitatively using ImageQuant 5.2 (Molecular Dynamics). The signals were either quantified after standardization with respect to actin-mRNA quantities or with the aid of ethidium bromide-stained 18s and 28s rRNA bands.

Isolation of Chromosomal DNA and Southern Hybridization

Chromosomal DNA of C. albicans was isolated as described (Millon et al., J. Clin. Microbiol., 32 (1994), 1115-1118), cleaved using EcoRI or PstI and separated on 0.8% agarose gels. The application to nylon membranes in 20×SSC and the hybridization with a gene-specific probe, which was labeled with [α-³²P]dCTP according to the random principle, was carried out according to standard protocols (Sambrook, 1989). The membranes were developed for 12 h using a phosphor screen (Molecular Dynamics). They were subsequently scanned and visualized as described for the Northern hybridization.

Results

The DNA Microarray Analysis Leads to the Identification of New Cell Wall Genes Regulated by RIM101 in the C. albicans

The cell wall of C. albicans protects the fungus from its environment and is of central importance for the host-pathogen interaction. It is known that the pH-dependent transcription factor RIM101 regulates two glycosidases which are involved in the cell wall biosynthesis of C. albicans (Muhlschlegel and Fonzi, Mol. Cell. Biol., 17 (1997), 5960-5967). For the identification of further cell wall genes regulated by RIM101, a transcription analysis was carried out using a cell wall-specific DNA microarray. The chip experiments were carried out using a Δrim101 mutant strain and the strain RIM101-1426, which overexpresses the C-terminal truncated, dominant active protein Rim101-1426p. The mutant strains were compared with wild-type cells in YPD medium at a temperature of 30° C. and a pH of 7.4 or 4.5. For the detection of the gene regulation events caused by the pH changes, all strains were grown under conditions which favor the formation of a blastospore morphology. None of the strains therefore showed filamentous growth when the cells were harvested for RNA isolation after culturing for six hours.

The Genes RBR1, RBR2 and RBR3 are Repressed by Rim101p

The data obtained show that a number of open laser frames not characterized up to now are repressed by Rim101p, to which RBR1 (orf6.6747), RBR2 (orf6.6744) and RBR3 (orf6.1159) belong. For the confirmation of the microarray data and for the quantification of the transcription of these genes, a Northern blot analysis was carried out. The results are shown in FIG. 1b. RBR1, RBR2 and RBR1 were acid-expressed, no significant expression in the wild-type being detected at a neutral pH. The RBR genes were repressed by dominantly active RIM101-1426 at an acidic pH and strongly highly regulated under all conditions in Δrim101 mutant strains (see FIG. 1b and FIG. 4). According to an in silico investigation, all three RIM101-repressed genes have an N-terminal signal sequence comprising approximately 20 amino acid for transport into the endoplasmic reticulum and a C-terminal hydrophobic transmembrane domain. All RBR protein sequences likewise comprise a hydrophobic N-terminal domain. The transmembrane domains are characteristic of GPI-anchored cell wall proteins. To the transmembrane domains is connected a sequence situated upstream having an omega site, which defines the cleavage site for the addition of the GPI anchor as well as further determinants which define the location either in the cell wall or in the plasma membrane (Caro et al., Yeast, 13 (1997), 1477-1489; De Groot et al., Yeast, 20 (2003), 781-796; Frieman and Cormack, Mol. Microbiol., 50 (2003), 883-896). The proteins Rbr1p (111 amino acids) and Rbr2p (168 amino acids) have potential cleavage sites in positions 81 and 143 respectively. The protein Rbr3p (560 amino acids) shows a potential cleavage site in position 540. Moreover, the protein sequence, with the exception of the C- and N-terminal hydrophobic sequences, shows a number of sites having a high O-beta-glycosylation potential, which is typical for fungal cell wall proteins (http://www.cbs.dtu.dk/services/YinoYang).

A search of pro- and eukaryotic genome databases at the National Center for Biotechnology Information (http://www.ncbi.nlm.nih.gov/BLAST) showed that the RBR genes according to the invention show no significant homology to genes of S. cerevisiae or other organisms. It was seen that Rbr1p is approximately 40% identical with Hyr1p of C. albicans, a GPI-anchored, nonessential cell wall protein which very often occurs in hyphae (Bailey et al., J. Bacteriol., 178 (1996), 5353-5360).

The Δrbr1 Mutant Strain Shows a pH-Dependent Filamentation Defect

Since RBR1 showed the strongest expression compared with the other RIM101-repressed genes, this gene was characterized in more detail. The open laser frame (orf6.6747), which encodes Rbr1p, has a length of 336 bp and encodes a GPI-anchored protein of only 111 amino acids. For the functional characterization of the cell wall protein Rbr1p in C. albicans, three independent Δrbr1 mutant strains were constructed by means of transformation and homologous recombination of CA14 using the URA3 flipper cassette (Morschhauser et al., 1999). The strains were routinely first selected on SC-uri medium and in a second step, after induction of the FLP recombinase, on ura3⁻ colonies. Homozygous Δrbr1 mutants were reverted either using only the URA3 gene or using URA3 and RBR1 under the control of its native promoter. Heterozygous and homozygous Δrbr1 mutants showed no growth defects under the media conditions used. Southern and Northern blot analyses confirmed the genotype and the RBR1 transcription. Since RBR1 is mainly expressed at a low pH (compare FIG. 1b), under these conditions possible changes in the growth and in the resistance to cell wall stress and filamentation defects are to be expected. Therefore the growth and the filament induction of the Δrbr1 mutants was investigated on different solid media at 25° C., 30° C. and 37° C. in the presence of hydrogen peroxide, calcofluor white and 0.3 M MaCl. On media containing 2% agar, no distinct phenotype was observed, the Δrbr1 strains, however, showing a filamentation defect on soft agar containing 0.3% agar. In a three-day incubation on M-199 soft agar having a pH of 4.5, Δrbr1 showed no filamentous growth (compare FIG. 2), whereas with the wild-type and the RBR1 revertant filamentation was induced after 24 hours at 30° C. Under the same conditions and at a pH of 7.4, the Δrbr1 strain induced no filaments after 24 hours (data not shown). This defect in the filament induction points to the fact that RBR1 is needed in an acidic environment, in particular in the cell wall of C. albicans. This agrees with the expression profile of RBR1 (compare FIG. 1b). After 5 days on an acidic M-199 soft agar at 30° C., the Δrbr1 mutant strains also began to form hyphae, which is possibly to be attributed to nutrient deficiency not connected therewith. If the test was carried out in liquid media, no difference in the growth or the filamentation rate was found between the wild-type and the Δrbr1 mutant (data not shown), which points to the fact that surface-mediated effects could be responsible for the phenotype observed.

NRG1 Activates the Expression of RBR1

Since Rbr1p appears to be involved in the dimorphism switch, the question should be clarified of whether in addition to Rim101p other transcription regulators of the morphogenesis in C. albicans are also involved in RBR1 regulation. Therefore the expression of RBR1 in the mutant strains Δefg1, Δnrg1 and Δtup1 was investigated at a pH of 4.5 at 25° C. and a pH of 7.4 at 37° C. in hyphae-induced media (FIG. 3). It turned out here that the expression of RBR1 in the mutant Δnrg1 at a pH of 4.5 was strongly reduced, whereas in the mutants Δefg1 and Δtup1 transcript quantities corresponding to the wild-type were detected. At a neutral pH, no significant RBR1 expression was found, either in the wild-type or in one of the deletion mutants tested. These data point to the fact that Nrg1p directly or indirectly activates RBR1 transcription, whereas Efg1p and Tup1p were not involved in RBR1 regulation under the conditions tested.

TABLE 1
C. albicans strains used

| Strain | Former name | Genotype | Reference |
|---|---|---|---|
| Wild-type | SC5314 | Clinical isolate | (Gillum et al., 1984) |
| CA/4 | CA/4 | ura3::imm434/ura3::imm434 | (Fonzi & Irwin, 1993) |
| Δrim101 | CAF3-X | ura3::imm434/ura3::imm434 | (El Barkani et al., 2000) |
| | | rim101::hisG/rim101::hisG/rim101::hisG::URA3 | |
| RIM101-1426 | CAF3-16-2 | ura3::imm434/ura3::imm434 | (El Barkani et al., 2000) |
| | | RIM101/RIM101-(pBSK+ -RIM101-1426-URA3)_{n>2} | |
| Δefg1 | HLC52 | ura3::imm434/ura3::imm434 | (Lo et al., 1997) |
| | | efg1::hisG/efg1::hisG::URA3 | |
| Δnrg1 | MMX3 | ura3::imm434/ura3::imm434 | (Murad et al., 2001b) |
| | | nrg1::hisG/nrg1::hisG::URA3 | |
| Δtup1 | BCA2-9 | ura3::imm434/ura3::imm434 | (Murad et al., 2001b) |
| | | tup1::hisG/tup1::hisG::URA3 | |
| Δrbr1 | HL12-2 | ura3::imm434/ura3::imm434 | ⁺ |

| | | |
|---|---|---|
| | | rbr1::FRT*/rbr1::FRT rpn10::URA3 |
| Δrbr1-RBR1 | HL13-6 | ura3::imm434/ura3::imm434 |
| | | rbr1::FRT/rbr1::FRT rpn10::RBR1-URA3 |

*FRT designates the FLP recognition sites which remains after excision of the URA3-FRT flipper cassette
⁺Mutant strains were constructed 3 times independently of one another.

1. A nucleic acid molecule which encodes a cell wall protein necessary for the hyphae development of a pathogenic fungal organism, selected from the group consisting of:
a) a nucleic acid molecule having one of the nucleotide sequences shown in SEQ ID No. 1, SEQ ID No. 3 or SEQ ID No. 5,
b) a nucleic acid molecule having a nucleotide sequence which encodes a protein having one of the amino acid sequences shown in SEQ ID No. 2, SEQ ID No. 4 or SEQ ID No. 6,
c) a nucleic acid molecule having a nucleotide sequence which over its entire length shows a homology of at least 80% to a nucleotide sequence of one of the nucleic acid molecules of a) or b), and
d) a nucleic acid molecule having a nucleotide sequence which is complementary to a nucleotide sequence of one of the nucleic acid molecules of a) to c).

2. The nucleic acid molecule as claimed in claim 1, the nucleic acid molecule having the sequence of the RBR1 gene of Candida albicans.

3. The nucleic acid molecule as claimed in claim 1, the nucleic acid molecule having the sequence of the RBR2 gene of C. albicans.

4. The nucleic acid molecule as claimed in claim 1, the nucleic acid molecule having the sequence of the RBR3 gene of C. albicans.

5. The nucleic acid molecule as claimed in claim 2, the RBR1 gene being a pH— and/or temperature-regulated gene.

6. The nucleic acid molecule as claimed in claim 2 or 5, the expression of the RBR1 gene being activated by the repressor Nrg1p and repressed by the transcription factor Rim101p.

7. The nucleic acid molecule as claimed in one of claims 1 to 6, the nucleic acid molecule being present as a DNA, RNA, PNA or LNA molecule or as a mixed form thereof.

8. A fragment of a nucleic acid molecule as claimed in one of claims 1 to 7, wherein it can inhibit the expression of a cell wall protein of a pathogenic fungal organism in antisense orientation to a promoter in a host cell and comprises at least 10 nucleotides.

9. The fragment as claimed in claim 8, wherein it comprises at least 15, in particular at least 25, preferably at least 50 and particularly preferably at least 100 nucleotides.

10. A vector, which comprises at least one nucleic acid molecule as claimed in one of claims 1 to 7 and/or at least one nucleic acid fragment as claimed in claim 8 or 9 under the functional control of at least one expression regulation element which guarantees the transcription of the nucleic acid into a translatable RNA and/or the translation of the RNA into a protein.

11. The vector as claimed in claim 10, wherein the vector is a plasmid, cosmid, bacteriophage or virus.

12. The vector as claimed in claimed 10 or 11, wherein the regulation element is a promoter, enhancer, silencer, 3′-transcription terminator or a ribosome binding site.

13. The vector as claimed in one of claims 10 to 12, the vector having a signal sequence for the transport of the expressed protein into a cell organelle, a cell compartment, into the extracellular space or out of the cell.

14. The vector as claimed in one of claims 10 to 13, wherein the nucleic acid molecule or fragment thereof is arranged in an antisense orientation to the at least one regulation element.

15. A host cell, wherein it contains at least one vector as claimed in one of claims 10 to 14.

16. The host cell as claimed in claim 14, wherein the host cell is a prokaryotic or eukaryotic cell, wherein the host cell is a bacterial cell, yeast cell, insect cell or mammalian cell.

17. The host cell as claimed in claim 15 or 16.

18. The host cell as claimed in one of claims 15 to 17, wherein the host cell is a C. albicans cell.

19. The host cell as claimed in claim 18, wherein the C. albicans cell comprises a vector in which a nucleic acid molecule having one of the nucleotide sequences shown in SEQ ID No. 1, SEQ ID No. 3 or SEQ ID No. 5 or having a nucleotide sequence which encodes a protein having one of the amino acid sequences shown in SEQ ID No. 2, SEQ ID No. 4 or SEQ ID No. 6 is arranged in antisense orientation to at least one regulation element.

20. A method for the production of a cell wall protein necessary for the hyphae development of a pathogenic fungal organism, in particular of the Rbr1p, Rbr2p or Rbr3p protein of C. albicans, comprising the culturing of a host cell as claimed in one of claims 15 to 18 in a suitable culture medium under conditions which allow the expression of the cell wall protein, and the obtainment of the expressed cell wall protein from the cell or from the medium.

21. A protein which has the amino acid sequence shown in SEQ ID No. 2 and which is encoded by the nucleic acid sequence shown in SEQ ID No. 1.

22. The protein as claimed in claim 21, the protein being the Rbr1p protein of C. albicans.

23. A protein which has the amino acid sequence shown in SEQ ID No. 4 and which is encoded by the nucleic acid sequence shown in SEQ ID No. 3.

24. The protein as claimed in claim 23, the protein being the Rbr2p protein of C. albicans.

25. A protein which has the amino acid sequence shown in SEQ ID No. 6 and which is encoded by the nucleic acid sequence shown in SEQ ID No. 5.

26. The protein as claimed in claim 25, the protein being the Rbr3p protein of C. albicans.

27. The protein as claimed in one of claims 21 to 26, produced by the method as claimed in claim 20.

28. An antibody which specifically recognizes a protein as claimed in one of claims 21 to 27 and binds thereto.

29. The antibody as claimed in claim 28, the antibody being a monoclonal or a polyclonal antibody.

30. An antibody which specifically recognizes an antibody as claimed in claim 28 or 29 and binds thereto.

31. A method for the characterization and/or for the detection of the hyphae stage of Candida cells or cells of pathogenic fungal organisms which are related to Candida, comprising the incubation of the cells or cell fractions thereof with an agent for the identification of the cell wall protein Rbr1p, Rbr2p and/or Rbr3p, of a homologous protein and/or of a fragment thereof, the detection of the protein or of a fragment thereof indicating the presence of the virulent hyphae stage of the cells.

32. The method as claimed in claim 31, the Candida cells to be characterized being cells of C. albicans, C. tropicalis, C. krusei, C. parapsilosis, C. guilliermondii, C. glabrata, C. dubliniensis or C. lusitaniae.

33. The method as claimed in claim 31, the cells to be characterized being cells of pathogenic fungal organisms which are related to Candida, or cells of a Trichosporon species or of a Blastoschizomyces species.

34. The method as claimed in one of claims 31 to 33, the cells to be characterized being present in a biological sample.

35. The method as claimed in one of claims 31 to 33, the cells to be characterized being cells isolated from a biological sample and enriched intact cells.

36. The method as claimed in one of claims 31 to 33, isolated cell fractions being employed for the characterization which are obtainable by cell disruption and fractionation of Candida cells or cells of species related to Candida and comprise at least one cell wall fraction.

37. The method as claimed in one of claims 31 to 36, the agent employed for the identification of the protein Rbr1p, Rbr2p or Rbr3p, of a homologous protein thereof or of a fragment thereof being an immunological agent.

38. The method as claimed in claim 37, the immunological agent being an antiserum directed against the protein Rbr1p, Rbr2p or Rbr3p, an antibody as claimed in claim 28 or 29 directed against the protein Rbr1p, Rbr2p or Rbr3p or a fragment thereof or a complex thereof.

39. The method as claimed in claim 38, the antibody having a label selected from the group consisting of a dye label, a radiolabel, a fluorescent label, a chemiluminescent label or an enzyme inducing a measurable reaction.

40. A method for the detection of a Candida infection and/or of an infection by pathogenic organisms related to Candida in a biological sample obtained from a human or animal organism, the presence of the protein Rbr1p, Rbr2p and/or Rbr3p, of a homologous protein thereof and/or of a fragment thereof in the biological sample and/or in the cell wall of Candida cells or cells of pathogenic organisms related to Candida optionally contained in the biological sample being detected, comprising
a) the incubation of the biological sample with an agent for the identification of the protein Rbr1p, Rbr2p and/or Rbr3p, a homologous protein thereof and/or a fragment thereof and
b) the detection of the interaction of the identification means with the protein or fragment thereof.

41. The method as claimed in claim 40, the Candida cells being cells of C. albicans, C. tropicalis, C. krusei, C. parapsilosis, C. guilliermondii, C. glabrata, C. dubliniensis or C. lusitaniae.

42. The method as claimed in claim 40, the cells of pathogenic fungal organisms which are related to Candida being cells of a Trichosporon species or a Blastoschizomyces species.

43. The method as claimed in one of claims 40 to 42, the biological sample being a skin or mucous membrane swab, an organ biopsy, a tissue biopsy, a body fluid, a body secretion, stool or a rinse from cavities or hollow organs.

44. The method as claimed in claim 43, the body fluid being sputum, urine, pleural effusion, spinal fluid, lymph or blood.

45. The method as claimed in claim 44, the blood being present as an unpurified blood sample, blood plasma or blood serum.

46. The method as claimed in claim 44 or 45, invasive candidiasis being detected by the detection of the protein Rbr1p, Rbr2p or Rbr3p or of a fragment thereof in blood or in the cell wall of Candida cells contained in the blood.

47. The method as claimed in one of claims 40 to 46, the agent employed for the identification of the protein Rbr1p, Rbr2p or Rbr3p being an immunological agent.

48. The method as claimed in claim 47, the immunological agent being an antiserum directed against the protein Rbr1p, Rbr2p or Rbr3p, an antibody as claimed in claim 28 or 29 directed against the protein Rbr1p, Rbr2p or Rbr3p, or a fragment thereof or a complex thereof.

49. The method as claimed in claim 47 or 48, the antibody having a label selected from the group consisting of a dye label, a radiolabel, a fluorescent label, a chemiluminescent label or an enzyme inducing a measurable reaction.

50. A method for the discovery and identification of substances having therapeutic action against diseases which are caused by Candida species or pathogenic fungal species which are related to Candida, a substance to be tested being brought into contact in a suitable medium with at least one agent selected from the group consisting of a nucleic acid molecule as claimed in one of claims 1 to 7, a nucleic acid fragment as claimed in claim 8 or 9, a vector as claimed in one of claims 10 to 14, a host cell as claimed in one of claims 15 to 19, a protein as claimed in one of claims 21 to 27 or an antibody as claimed in one of claims 28 to 30, and an interaction between the substance to be tested and the agent being detected.

51. A diagnostic composition comprising a nucleic acid molecule as claimed in one of claims 1 to 7, a nucleic acid fragment as claimed in claim 8 or 9, a vector as claimed in one of claims 10 to 14, a host cell as claimed in one of claims 15 to 19, a protein as claimed in one of claims 21 to 27 and/or an antibody as claimed in one of claims 28 to 30.

52. A pharmaceutical composition comprising a nucleic acid molecule as claimed in one of claims 1 to 7, a nucleic acid fragment as claimed in claim 8 or 9, a vector as claimed in one of claims 10 to 14, a host cell as claimed in one of claims 15 to 19, a protein as claimed in one of claims 21 to 27, an antibody as claimed in one of claims 28 to 30 and/or a substance which was identified by means of the method as claimed in claim 50.

53. The pharmaceutical composition as claimed in claim 52, it being a vaccine which contains a protein as claimed in one of claims 21 to 27 and which is suitable for the active immunization of a human or animal body against a Candida infection.

54. The pharmaceutical composition as claimed in claim 52, it being a vaccine which contains an antibody as claimed in claim 28 or 29 and which is suitable for the passive immunization of a human or animal body against a Candida infection.

55. The pharmaceutical composition as claimed in claim 53 or 54, the vaccine being present as a lyophilizate.

56. The pharmaceutical composition as claimed in claim 53 or 54, the vaccine being present as an aqueous colloidal solution or suspension.

57. The pharmaceutical composition as claimed in one of claims 53 to 56, additionally containing at least one adjuvant.

58. A kit for the in vitro identification of the cell wall protein Rbr1p, Rbr2p and/or Rbr3p of Candida species or of a pathogenic organism related to Candida and/or for the in vitro detection of the virulence of Candida cells or of cells of an organism which is related to Candida, comprising at least one container having an antibody which specifically recognizes the protein Rbr1p, Rbr2p or Rbr3p or a fragment thereof and binds thereto.

59. The kit as claimed in claim 58, comprising a second container having the isolated and purified protein Rbr1p, Rbr2p and/or Rbr3p of C. albicans.

60. The use of a nucleic acid molecule as claimed in one of claims 1 to 7, of a nucleic acid fragment as claimed in claim 8 or 9, of a vector as claimed in one of claims 10 to 14, of a host cell as claimed in one of claims 15 to 19, of a protein as claimed in one of claims 21 to 27 or of an antibody as claimed in one of claims 28 to 30 for the diagnosis of diseases of a human or animal organism which are caused by Candida species or pathogenic fungal organisms which are related to Candida.

61. The use of a nucleic acid molecule as claimed in one of claims 1 to 7, of a nucleic acid fragment as claimed in claim 8 or 9, of a vector as claimed in one of claims 10 to 14, of a host cell as claimed in one of claims 15 to 19, of a protein as claimed in one of claims 21 to 27 or of an antibody as claimed in one of claims 28 to 30 for the production of a diagnostic composition for the diagnosis of diseases of a human or animal organism which are caused by Candida species or pathogenic fungal organisms which are related to Candida.

62. The use of an agent which decreases or inhibits the expression and/or activity of the cell wall protein Rbr1p, Rbr2p, Rbr3p and/or of a homologous protein thereof as an active compound for the treatment and/or prevention of diseases of a human or animal organism which are caused by Candida species or pathogenic fungal organisms which are related to Candida.

63. The use of an agent which decreases or inhibits the expression and/or activity of the cell wall protein Rbr1p, Rbr2p, Rbr3p and/or of a homologous protein thereof as an active compound for the production of a pharmaceutical composition for the treatment and/or prevention of diseases of a human or animal organism which are caused by Candida species or pathogenic fungal organisms which are related to Candida.

64. The use as claimed in claim 62 or 63, the agent being selected from the group consisting of a nucleic acid molecule as claimed in one of claims 1 to 7, a nucleic acid fragment as claimed in claim 8 or 9, a vector as claimed in one of claims 10 to 14, a host cell as claimed in one of claims 15 to 19, a protein as claimed in one of claims 21 to 27, an antibody as claimed in one of claims 28 to 30 or a substance which was identified by means of the method as claimed in claim 50.

65. The use of a nucleic acid molecule as claimed in one of claims 1 to 7, of a nucleic acid fragment as claimed in claim 8 or 9, of a vector as claimed in one of claims 10 to 14, of a host cell as claimed in one of claims 15 to 19, of a protein as claimed in one of claims 21 to 27 or of an antibody as claimed in one of claims 28 to 30 for the identification and/or for the detection of substances which inhibit the expression or activity of the Rbr1p protein in a pathogenic fungal organism and are suitable as an active compound for the production of a pharmaceutical composition for the control of complaints caused by Candida species.

66. The use of a nucleic acid molecule having one of the nucleotide sequences shown in SEQ ID No. 1, SEQ ID No. 3 or SEQ ID No. 5, of a nucleic acid molecule having a nucleotide sequence which encodes a protein having one of the amino acid sequences shown in SEQ ID No. 2, SEQ ID No. 4 or SEQ ID No. 6, or of a fragment thereof for the isolation of a homologous nucleic acid which encodes the Rbr1p protein, the Rbr2p protein or Rbr3p protein of C. tropicalis, C. krusei, C. parapsilosis, C. guilliermondii, C. glabrata, C. dubliniensis, C. lusitaniae, of a Trichosporon species, of a Blastoschizomyces species or of another fungal pathogenic organism.

67. The use of an antibody as claimed in claim 28 or 29 for the characterization and/or for the detection of the virulent hyphae stage of Candida cells.
